SEQUENCE LISTING (1 sequence)

SEQ ID NO: 1 - DNA, 22 bp, Artificial Sequence
  Oligonucleotide for shRNA of Stk11
aggtcaagatcctcaagaagaa

(19) United States
(12) Patent Application Publication
(10) Pub. No.: US 2019/0046478 A1
(43) Pub. Date: Feb. 14, 2019

(54) BIOLOGICAL MARKER FOR IDENTIFYING CANCER PATIENTS FOR TREATMENT WITH A BIGUANIDE

(71) Applicant: THE ROYAL INSTITUTION FOR THE ADVANCEMENT OF LEARNING/MCGILL UNIVERSITY, Montreal (CA)

(72) Inventor: Russell Graham JONES, Beaconsfield (CA)

(21) Appl. No.: 16/059,224

(22) Filed: Aug. 9, 2018

Related U.S. Application Data
(60) Provisional application No. 62/543,879, filed on Aug. 10, 2017.

(51) Int. Cl. A61K 31/155; A61P 35/00; C12Q 1/6886; C12Q 1/6806

(52) U.S. Cl. CPC A61K 31/155; A61P 35/00; C12Q 1/6886; A61K 31/53; C12Q 2600/112; C12Q 2600/106; C12Q 2600/178; C12Q 1/6806

(57) ABSTRACT

The disclosure features a method of determining whether a patient diagnosed as having cancer is likely to respond to treatment with an effective amount of a biguanide. This method includes (a) determining an expression level of miR-17˜92 in a sample obtained from the patient; and (b) comparing the expression level to a reference expression level of miR-17˜92, where an increased expression level of miR-17˜92 in the sample as compared to a reference expression level identifies the patient as one who is likely to respond to treatment including administration of the effective amount of the biguanide. Also featured are methods of treating patients having a cancer that is likely to respond to treatment with an effective amount of a biguanide.

### CROSS-REFERENCE TO RELATED APPLICATIONS

The present application claims priority from U.S. provisional patent application 62/543,879 filed on Aug. 10, 2017 and herewith incorporated in its entirety.

### STATEMENT REGARDING THE SEQUENCE LISTING

The sequence listing associated with this application is provided in text format in lieu of a paper copy and is hereby incorporated by reference into the specification. The name of the text file containing the sequence listing is SequenceListing. The text file is 1 KB, was created on Aug. 9, 2018 and is being submitted electronically via EFS-Web.

### FIELD OF THE INVENTION

The present disclosure is directed to methods for identifying patients who may benefit from treatment with a biguanide and to treatment of these patients.

### BACKGROUND OF THE INVENTION

Biomarkers can be useful for identifying patients who are likely to respond to a given therapy, for selecting the right drug for the right patient and to avoid unnecessary treatment. There is an unmet need for biomarkers that are useful for tailoring a therapy to a particular patient as well as for stratifying patients.

### SUMMARY OF THE INVENTION

The present disclosure provides methods for determining if a subject diagnosed with cancer is likely to respond to treatment with an effective amount of a biguanide. The inventors discovered that the miR-17˜92 biomarker is affective at sensitizing cancer cells to biguanide treatment as a consequence of liver kinase B1 (LKB1) inhibition. As such, a subject whose cancer (e.g., a bladder cancer, a breast cancer, a colon cancer, a rectal cancer, a uterine cancer, a kidney cancer, leukemia, a liver cancer, a lung cancer, a skin cancer, a hematopoietic system cancer (such as lymphoma), a pancreatic cancer, a prostate cancer, a gastric cancer, a brain cancer, and a thyroid cancer) has increased expression of miR-17˜92, relative to a reference level, is likely to benefit from biguanide treatment, such as administration of Compound 1 described herein.

As such, the first aspect of the present disclosure features a method of determining whether a patient diagnosed as having cancer is likely to respond to treatment with an effective amount of a biguanide. This method includes (a) determining an expression level of miR-17˜92 in a sample obtained from the patient; and (b) comparing the expression level to a reference expression level of miR-17˜92, where an increased expression level of miR-17˜92 in the sample as compared to a reference expression level identifies the patient as one who is likely to respond to treatment including administration of the effective amount of the biguanide.

In one embodiment of the first aspect of the disclosure, the method further includes (c) informing the patient that he or she has an increased likelihood of being responsive to treatment with the effective amount of the biguanide.

In the second aspect, the disclosure features a method for selecting a therapy for a particular patient in a population of patients being considered for therapy. This method includes (a) detecting expression of miR-17˜92 in a sample obtained from the patient prior to administration of an effective amount of a biguanide; (b) comparing the expression level of miR-17˜92 to a reference expression level of miR-17˜92, where an increase in the level of expression of miR-17˜92 in the patient sample relative to the reference level identifies a patient who is likely to respond to treatment with the effective amount of the biguanide; and (c) selecting a therapy including an effective amount of a biguanide if the patient is identified as likely to respond to treatment with the therapy and recommending to the patient the selected therapy.

In one embodiment of the second aspect of the disclosure the method further includes (d) administering the selected therapy to the patient.

In other embodiments of the disclosure, the expression level of miR-17˜92 is increased at least one and a half-fold or two-fold relative to the reference expression level. In further embodiments the expression level of miR-17˜92 is increased at least three-fold, four-fold, five-fold, six-fold, seven-fold, eight-fold, nine-fold, or ten-fold relative to the reference expression level.

In further embodiments of the first and second aspects of the disclosure, the patient has an increased expression level of miR-17˜92 relative to the reference expression level and the method further includes administering to the patient the effective amount of the biguanide.

In a third aspect, the disclosure features a method of treating cancer in a patient. This method includes administering to the patient an effective amount of a biguanide, where prior to treatment, an expression level of miR-17˜92 in a sample obtained from the patient has been determined to be increased relative to a reference expression level.

In embodiments of the first three aspects of the disclosure, the reference expression level is (i) the expression level of miR-17˜92 in a reference population; or (ii) a pre-assigned expression level for miR-17˜92.

In other embodiments of the first three aspects of the disclosure, the miR-17˜92 expression level is determined using quantitative polymerase chain reaction (qPCR).

In further embodiments of the first three aspects of the disclosure, the cancer is selected from the group consisting of colon cancer, lung cancer, lymphoma and hematopoietic system cancer.

In one embodiment of the third aspect of the disclosure, the effective amount of the biguanide includes a N1-cyclic amine-N5-substituted biguanide derivative compound of Formula I or a pharmaceutically acceptable salt thereof:

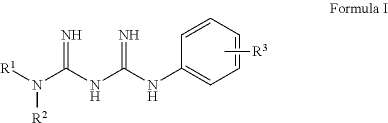

where R¹ and R² are taken together with nitrogen to which they are attached to form 3- to 8-membered heterocycloalkyl selected from the group consisting of azetidinyl, pyrrolidinyl, piperidinyl, piperazinyl, azepanyl and aziridinyl, where the heterocycloalkyl is unsubstituted or substituted with at least one substituent independently selected from the group consisting of halogen, hydroxy and C₁₋₆ alkyl;

R³ is unsubstituted or substituted and is selected from the group consisting of unsubstituted hydroxy, substituted C₁₋₆ alkyl, substituted C₁₋₆ alkoxy, unsubstituted or substituted C₁₋₆ alkylthio, unsubstituted or substituted amino, unsubstituted or substituted amide, unsubstituted or substituted sulfonamide, nitro, unsubstituted or substituted heteroaryl, cyano, sulfonic acid, and unsubstituted or substituted sulfamoyl, and

where the substituted R³ has at least one substituent selected from the group consisting of halogen, hydroxy and C₁₋₆ alkyl.

In particular embodiments of the third aspect of the disclosure, the biguanide includes an N1-cyclic amine-N5-substituted biguanide derivative compound of Formula I or a pharmaceutically acceptable salt thereof:

where R¹ and R² are taken together with nitrogen to which they are attached to form 3- to 7-membered heterocycloalkyl selected from the group consisting of azetidinyl, pyrrolidinyl, piperidinyl, piperazinyl, azepanyl and aziridinyl, where the heterocycloalkyl is unsubstituted or substituted with at least one substituent independently selected from the group consisting of halogen, hydroxy and C₁₋₆ alkyl;

R³ is unsubstituted or substituted and is selected from the group consisting of unsubstituted hydroxy, substituted C₁₋₆ alkyl, substituted C₁₋₆ alkoxy, unsubstituted or substituted C₁₋₆ alkylthio, unsubstituted or substituted amino, unsubstituted or substituted amide, unsubstituted or substituted sulfonamide, nitro, unsubstituted or substituted heteroaryl, cyano, sulfonic acid, and unsubstituted or substituted sulfamoyl, and

where the substituted R³ has at least one substituent selected from the group consisting of halogen, hydroxy and C₁₋₆ alkyl.

In another embodiment of the third aspect of the disclosure, the compound of Formula I is N1-pyrrolidine-N5-(3-trifluoromethoxy)phenyl biguanide, or a pharmaceutically acceptable salt thereof.

In further embodiments of the third aspect of the disclosure, the pharmaceutically acceptable salt is an acid addition salt of an acid selected from the group consisting of formic acid, acetic acid, propionic acid, lactic acid, butyric acid, isobutyric acid, trifluoroacetic acid, malic acid, maleic acid, malonic acid, fumaric acid, succinic acid, succinic acid monoamide, glutamic acid, tartaric acid, oxalic acid, citric acid, glycolic acid, glucuronic acid, ascorbic acid, benzoic acid, phthalic acid, salicylic acid, anthranyl acid, benzensulfonic acid, p-toluenesulfonic acid, methanesulfonic acid, dichloroacetic acid, aminooxy acetic acid, hydrochloric acid, hydrobromic acid, sulfuric acid, phosphoric acid, nitric acid, carbonic acid, and boric acid.

In a fourth aspect, the disclosure features a kit for determining whether a patient diagnosed as having cancer is likely to respond to treatment with an effective amount of a biguanide, the kit including instructions for use of qPCR to determine an expression level of miR-17˜92, where an increase in the expression level of miR-17˜92 relative to a reference level expression level of miR-17˜92 indicates that the patient is likely to respond to treatment with the effective amount of the biguanide.

### Definitions

The term “biguanide” refers to a class of compounds with basic properties, made from two guanidine molecules. Biguanides of the present disclosure include compounds of Formula I,

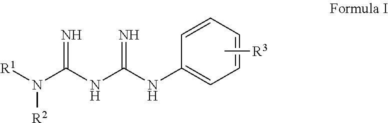

where R¹ and R² are taken together with nitrogen to which they are attached to form 3- to 8-membered heterocycloalkyl selected from azetidinyl, pyrrolidinyl, piperidinyl, piperazinyl, azepanyl and aziridinyl, where the heterocycloalkyl is unsubstituted or substituted with at least one substituent independently selected from halogen, hydroxyl, and C₁₋₆ alkyl; R³ is unsubstituted or substituted and is selected from unsubstituted hydroxy, substituted C₁₋₆ alkyl, substituted C₁₋₆ alkoxy, unsubstituted or substituted C₁₋₆ alkylthio, unsubstituted or substituted amino, unsubstituted or substituted amide, unsubstituted or substituted sulfonamide, nitro, unsubstituted or substituted heteroaryl, cyano, sulfonic acid, and unsubstituted or substituted sulfamoyl, and where the substituted R³ has at least one substituent selected from halogen, hydroxyl, and C₁₋₆ alkyl. An exemplary biguanide is Compound 1 (N1-pyrrolidine-N5-(3-trifluoromethoxy)phenyl biguanide).

As used herein, the term “cancer” refers to a condition characterized by abnormal cell growth. The terms “cancer cell,” “tumor cell,” and “tumor” refer to an abnormal cell, mass, or population of cells that result from excessive division that may be malignant or benign and all pre-cancerous and cancerous cells and tissues. Examples of cancer include, but are not limited to, a bladder cancer, a breast cancer, a colon cancer, a rectal cancer, a uterine cancer, a kidney cancer, leukemia, a liver cancer, a lung cancer, a skin cancer, a hematopoietic system cancer (such as a lymphoma), a pancreatic cancer, a prostate cancer, a gastric cancer, a brain cancer and a thyroid cancer.

“Increased expression level” refers to an increased expression or increased levels of a marker, e.g., a miR-17˜92 biomarker, in an individual relative to a control, such as an individual or individuals who do not have cancer (e.g., a bladder cancer, a breast cancer, a colon cancer, a rectal cancer, a uterine cancer, a kidney cancer, leukemia, a liver cancer, a lung cancer, a skin cancer, a hematopoietic system cancer (such as lymphoma), a pancreatic cancer, a prostate cancer, a gastric cancer, a brain cancer, or a thyroid cancer) (e.g., healthy individuals), an internal control (e.g., a reference biomarker), or a median expression level of the biomarker in samples from a group/population of subjects. The increased expression level may be one and a half-fold, two-fold, three-fold, four-fold, five-fold, six-fold, seven-fold, eight-fold, nine-fold, ten-fold, eleven-fold, twelve-fold, or fifteen-fold relative to the reference expression level.

The term “expression level” or “level of expression” are used interchangeably and generally refer to the amount of a polynucleotide, a peptide, or protein in a biological sample, e.g., a biomarker. “Expression” generally refers to the process by which gene-encoded information is converted into the structures present or operating in the cell. Therefore, according to the disclosure, “expression” of a gene may refer to transcription into a polynucleotide, translation into a protein, or even posttranslational modification of the protein. Fragments of the transcribed polynucleotide, the translated protein, or the post-translationally modified protein shall also be regarded as expressed whether they originate from a transcript generated by alternative splicing or a degraded transcript, or from a post-translational processing of the protein, e.g., by proteolysis. “Expressed genes” include those that are transcribed into a polynucleotide as mRNA and then translated into a protein, and also those that are transcribed into RNA but not translated into a protein.

As used herein, the term “reference expression level” refers to an expression level against which another expression level, e.g., the expression level of miR-17˜92 in a sample from an individual is compared, e.g., to make a predictive, diagnostic, prognostic, and/or therapeutic determination. The reference expression level may be derived from expression levels in a reference population (e.g., the median expression level in a reference population, e.g., a population of patients having a cancer), a reference sample, and/or a pre-assigned value (e.g., a cut-off value which was previously determined to significantly (e.g., statistically significantly) separate a first subset of individuals who have been treated with an anti-cancer therapy (e.g., an anti-cancer therapy including a biguanide) in a reference population and a second subset of individuals who have been treated with a different anti-cancer therapy (or who have not been treated with the anti-cancer therapy) in the same reference population based on a significant difference between an individual's responsiveness to treatment with the anti-cancer therapy and an individual's responsiveness to treatment with the different anti-cancer therapy above the cut-off value and/or below the cut-off value). In some embodiments, the cut-off value may be the median or mean expression level in the reference population. In other embodiments, the reference level may be the top 40%, the top 30%, the top 20%, the top 10%, the top 5%, or the top 1% of the expression level in the reference population. It will be appreciated by one skilled in the art that the numerical value for the reference expression level may vary depending on the indication (e.g., a cancer (e.g., a bladder cancer, a breast cancer, a colon cancer, a rectal cancer, a uterine cancer, a kidney cancer, leukemia, a liver cancer, a lung cancer, a skin cancer, a hematopoietic system cancer (such as lymphoma), a pancreatic cancer, a prostate cancer, a gastric cancer, a brain cancer, and a thyroid cancer)), the methodology used to detect expression levels (e.g., qPCR).

The term “miR-17˜92,” also known as “oncomiR-1,” refers to a microRNA cluster (Mogilyansky et al., Cell Death Differ. 20(12):1603-1614 (2013)). The miR-17˜92 cluster is located in the locus of the non-protein-coding gene MIR17HG (the miR-17˜92 cluster host gene), also known as C13orf25. The miR-17˜92 cluster transcript spans 800 nucleotides out of MIR17HG's 7 kilobase pair (kb) and includes six miRNAs: miR-17, miR-18a, miR-19a, miR-20a, miR-19b-1, and miR-92a-1.

“Quantitative real-time polymerase chain reaction” or “qPCR” refers to a form of polymerase chain reaction (PCR) where the amount of PCR product is measured at each step in a PCR reaction. This technique is described by, for example, Cronin et al., Am. J. Pathol. 164(1):35-42 (2004), and Ma et al., Cancer Cell 5:607-616 (2004).

A “reference biomarker,” “reference sample,” “reference cell,” “reference tissue,” “control sample,” “control cell,” or “control tissue,” as used herein, refers to a marker, a sample, cell, tissue, standard, or level that is used for comparison purposes. In one embodiment, a reference level, reference sample, reference cell, reference tissue, control sample, control cell, or control tissue is obtained from a healthy and/or non-diseased part of the body (e.g., tissue or cells) of the same subject. For example, a reference sample, reference cell, reference tissue, control sample, control cell, or control tissue may be healthy and/or non-diseased cells or tissue adjacent to the diseased cells or tissue (e.g., cells or tissue adjacent to a tumor). In another embodiment, a reference sample is obtained from an untreated tissue and/or cell of the body of the same subject. In yet another embodiment, a reference sample, reference cell, reference tissue, control sample, control cell, or control tissue is obtained from a healthy and/or non-diseased part of the body (e.g., tissues or cells) of an individual who is not the subject. In even another embodiment, a reference sample, reference cell, reference tissue, control sample, control cell, or control tissue is obtained from an untreated tissue and/or cell of the body of an individual who is not the subject. In another embodiment, a reference sample, reference cell, reference tissue, control sample, control cell, or control tissue is obtained from a subject prior to administration of a therapy (e.g., a biguanide).

The term “sample,” as used herein, refers to a composition that is obtained or derived from a subject of interest that contains a cellular and/or other molecular entity that is to be characterized and/or identified, for example, based on physical, biochemical, chemical, and/or physiological characteristics. Samples include, but are not limited to, tissue samples, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, plasma, urine, cerebro-spinal fluid, saliva, sputum, tears, perspiration, mucus, tumor lysates and tissue culture medium, tissue extracts such as homogenized tissue, tumor tissue, cellular extracts, and combinations thereof.

A “therapeutically effective amount” refers to an amount of a therapeutic agent (used alone or in combination with a further therapy) to successfully treat or prevent the recurrence of a disease (e.g., a cancer (e.g., a bladder cancer, a breast cancer, a colon cancer, a rectal cancer, a uterine cancer, a kidney cancer, leukemia, a liver cancer, a lung cancer, a skin cancer, a hematopoietic system cancer (such as lymphoma), a pancreatic cancer, a prostate cancer, a gastric cancer, a brain cancer, and a thyroid cancer)) in a mammal. In the case of cancers, the therapeutically effective amount of the therapeutic agent may reduce the number of cancer cells, reduce the primary tumor size, inhibit (i.e., slow to some extent and preferably stop) cancer cell infiltration into peripheral organs, inhibit (i.e., slow to some extent and preferably stop) tumor metastasis, inhibit, to some extent, tumor growth, and/or relieve to some extent one or more of the symptoms associated with the disease. To the extent the drug may prevent growth and/or kill existing cancer cells, it may be cytostatic and/or cytotoxic. For cancer therapy, efficacy in vivo can, for example, be measured by assessing the duration of survival (e.g., overall survival or progression-free survival), time to disease progression (TTP), response rates (e.g., complete response (CR) and partial response (PR)), duration of response, and/or quality of life.

As used herein, a “pharmaceutical composition” or “pharmaceutical preparation” is a composition or preparation, having pharmacological activity or other direct effect in the mitigation, treatment, or prevention of disease, and/or a finished dosage form or formulation thereof and which is indicated for human use. A pharmaceutical composition may include an active ingredient and an excipient and/or adjuvant.

The term “treatment” (and grammatical variations thereof such as “treat” or “treating”) refers to clinical intervention in an attempt to alter the natural course of the individual being treated and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

A “substituted” group refers to a group in which at least one hydrogen atom is replaced with at least one non-hydrogen atom group, provided that the group satisfies the valence electron requirements and forms a chemically stable compound from the substitution. Unless explicitly described as “unsubstituted” in this specification, it should be understood that all substituents will be unsubstituted or substituted with another substituent.

The term “halogen” or “halo” refers to fluoro, chloro, bromo, and iodo.

The term “hydroxyl” refers to —OH.

The term “alkyl” refers to a linear and branched saturated hydrocarbon group generally having a specified number of carbon atoms (for example, 1 to 12 carbon atoms). Examples of alkyl groups include, without limitation, methyl, ethyl, n-propyl, i-propyl, n-butyl, s-butyl, i-butyl, t-butyl, pent-1-yl, pent-2-yl, pent-3-yl, 3-methylbut-1-yl, 3-methylbut-2-yl, 2-methylbut-2-yl, 2,2,2-trimethylethyl, n-hexyl, n-heptyl, and n-octyl. The alkyl may be attached to a parent group or a substrate at any ring atom, unless the attachment would violate valence electron requirements. Likewise, the alkyl group may include at least one non-hydrogen substituent unless the substitution would violate valence electron requirements. For example, the term “haloalkyl” refers to a group such as —CH₂(halo), —CH(halo)₂ or C(halo)₃, i.e., a methyl group in which at least one hydrogen atom is replaced with halogen. Examples of “haloalkyl” groups include, without limitation, trifluoromethyl, trichloromethyl, tribromomethyl, and triiodomethyl.

The term “alkoxy” refers to alkyl-O—, provided that the alkyl is the same as defined above. Examples of the alkoxy group include, without limitation, methoxy, ethoxy, n-propoxy, i-propoxy, n-butoxy, s-butoxy, t-butoxy, n-pentoxy, and s-pentoxy. The alkoxy may be attached to a parent group or a substrate at any ring atom, unless the attachment would violate valence electron requirements. Likewise, the alkoxy group may include at least one non-hydrogen substituent unless the attachment would violate valence electron requirements. For example, “haloalkoxy” refers to —O—CH₂(halo), —O—CH(halo)₂ or —O—C(halo)₃, i.e., a methyl group in which at least one hydrogen atom is replaced with halogen. Examples of “haloalkoxy” group include, without limitation, trifluoromethoxy, trichloromethoxy, tribromomethoxy, and triiodomethoxy.

The term “alkylthio” refers to alkyl-S—, provided that the alkyl is the same as defined above. Examples of the alkylthio group include, without limitation, methylthio, ethylthio, n-propylthio, i-propylthio, n-butylthio, s-butylthio, t-butylthio, n-pentylthio, and s-pentylthio. The alkylthio group may be attached to a parent group or a substrate at any ring atom, unless the attachment would violate valence electron requirements. Likewise, the alkylthio group may include at least one non-hydrogen substituent unless the attachment would violate valence electron requirements.

The term “cycloalkyl” refers to a saturated monocyclic and dicyclic hydrocarbon ring generally having the specified number of carbon atoms that include a ring (for example, C₃₋₈ cycloalkyl refers to a cycloalkyl group having 3, 4, 5, 6, 7 or 8 carbon atoms as a ring member). The cycloalkyl may be attached to a parent or substrate at any ring atom, unless the attachment would violate valence electron requirements. Likewise, the cycloalkyl group may include at least one non-hydrogen substituent unless the substitution would violate valence electron requirements. The term “heterocycloalkyl” refers to a monocyclic and dicyclic hydrocarbon ring having 3 to 12-membered ring atoms containing 1 to 3 heteroatoms independently selected from nitrogen, oxygen, and sulfur. The heterocycloalkyl may be attached to a parent or substrate at any ring atom, unless the attachment would violate valence electron requirements. Likewise, the heterocycloalkyl group may include at least one non-hydrogen substituent unless the substitution would violate valence electron requirements. Examples of the heterocycloalkyl group include, without limitation, aziridine, azetidine, imidazolyl, pyrrolyl, pyrrolidinyl, piperidyl, morpholinyl, piperazinyl, azepanyl, indolyl, and indolinyl.

The term “amino” refers to a —NH₂ group. The “amino” group may include at least one non-hydrogen substituent unless the substitution would violate valence electron requirements. For example, the term “dialkylamino” refers to —N(alkyl)₂, provided that the alkyl is the same as defined above. Examples of “dialkylamino” include, without limitation, dimethylamine, diethylamine, dipropylamine, and dibutylamine.

The term “amide” refers to —NH—C(O)—R′. Here, the residue R′ represents a lower alkyl having 1 to 6 carbon atoms. Examples of the “amide” group include, without limitation, acetamide, propanamide, and butanamide.

The term “sulfonamide” refers to —NH—S(O)₂—R′, provided that the residue R′ represents, for example, a lower alkyl having 1 to 6 carbon atoms. An example of a “sulfonamide” group is, without limitation, methylsulfonamide.

The term “aryl” refers to monovalent and bivalent aromatic groups, respectively including 5- and 6-membered monocyclic aromatic groups, and “heteroaryl” refers to monovalent and bivalent aromatic groups, respectively, including 5- and 6-membered monocyclic aromatic groups that contain 1 to 4 heteroatoms independently selected from nitrogen, oxygen, and sulfur. Examples of the “heteroaryl” group include, without limitation, furanyl, pyrrolyl, thiopheneyl, thiazolyl, isothiazolyl, imidazolyl, triazolyl, tetrazolyl, pyrazolyl, oxazolyl, isooxazolyl, pyrazinyl, pyrazinyl, pyridazinyl, pyrimidinyl, isoquinolinyl, carbazolyl, benzoxazolyl, benzodioxazolyl, benzothiazolyl, benzimidazolyl, benzothiophenyl, triazinyl, phthalazinyl, quinolinyl, indolyl, benzofuranyl, furinyl and indolizinyl.

The term “sulfamoyl” refers to —S(O)₂—NH₂, and the term “sulfonic acid” refers to —S(O)₂—OH.

### BRIEF DESCRIPTION OF THE DRAWINGS

FIG. 1A to FIG. 1D are a series of graphs showing: (FIG. 1A) Viability of ﬂ/ﬂ and Δ/Δ cells after 48 hour treatment with vehicle or phenformin; (FIG. 1B) Viability of ﬂ/ﬂ and Δ/Δ cells transduced with control of LKB1 shRNA following 48 hour phenformin treatment; (FIG. 1C) Eμ-Myc cells transduced with control (Ctrl) or miR-17˜92 (+17˜92) vectors treated with vehicle or phenformin for 48 hours and assessed for viability; (FIG. 1D) Ctrl and +17˜92 human Raji lymphoma cells treated with vehicle or phenformin for 48 hours and assessed for viability. Statistics for all figures are as follows: *, p<0.05; **, p<0.01; ***, p<0.001.

FIG. 2A to FIG. 2E are a series of graphs and images showing: (FIG. 2A-B) OCR and ECAR of Eμ-Myc cells before and after injection of 100 μM of either phenformin or Compound 1, drug injection is illustrated by dashed red line; (FIG. 2C-D) Percent reduction in OCR of Eμ-Myc cells 5 min and 260 min post-biguanide injection (phenformin (solid bars) or compound 1 (diagonal hatched bars) at a dose of 0.00045 (1), 0.0014 (2), 0.0041 (3), 0.0123 (4), 0.037 (5), 0.111 (6), 0.333 (7) or 1.0 mM (8)); (FIG. 2E) Eμ-Myc cells treated with vehicle, 100 μM phenformin, or 10 μM Compound 1 and immunobloted for pAMPK (T172) and total AMPK. Statistics for all figures are as follows: *, p<0.05; **, p<0.01; ***, p<0.001.

FIG. 3A to FIG. 3C are a series of graphs and images showing: (FIG. 3A) Viability of Ctrl (● and ▪) and +17˜92 (▴ and ▾) Eμ-Myc cells following 48 hours treatment with indicated doses of phenformin (● and ▴) or Compound 1 (▪ and ▾); (FIG. 2B) Viability of Ctrl (● and ▪) and +17˜92 (▴ and ▾) Raji cells following 48 hours treatment with indicated doses of phenformin (● and ▴) or Compound 1 (▪ and ▾); (FIG. 3C) Ctrl and +17˜92 Eμ-Myc cell treated with 100 μM phenformin (left) or 10 μM Compound 1 (right) for 2 hours before immunoblotting for pAMPK (T172) and total AMPK.

FIG. 4A to FIG. 4B are a series of graphs showing: (FIG. 4A) Ctrl (white) and +17˜92 (black) Eμ-Myc cells treated with vehicle, 100 μM phenformin or 10 μM Compound 1 for 2 hours before harvesting for GC-MS, data presented as percentage of metabolite abundance detected in vehicle treated cells; (FIG. 4B) Ctrl (white) and +17˜92 (black) Eμ-Myc cells treated for 2 hours with 100 μM phenformin or 10 μM Compound 1 before being incubated with uniformly labelled ¹³C-glutamine for an additional 2 hours of treatment and labelling, data presented as percent of total metabolite pool derived from ¹³C-glutamine. Statistics for all figures are as follows: *, p<0.05; **, p<0.01; ***, p<0.001.

FIG. 5A to FIG. 5E are a series of graphs showing: (FIG. 5A) Mitochondrial ROS in control (white bars) and +17˜92 cells (black bars) following 2 hours vehicle treatment (−), 100 μM phenformin treatment (P), or 10 μM Compound 1 treatment (I) as measured by MitoSox staining and flow cytometry, data presented as mean fluorescence intensity (MFI); (FIG. 5B) Ratios of GSH:GSSG in control (white bars) and +17˜92 cells (black bars) following 2 hours vehicle treatment (−), 100 μM phenformin treatment (P), or 10 μM Compound 1 treatment (I) as measured by LC-MS; (FIG. 5C) Ratios of NADP+/NADPH in control (white bars) and +17˜92 cells (black bars) following 2 hours vehicle treatment (−), 100 μM phenformin treatment (P), or 10 μM Compound 1 treatment (I) as measured by LC-MS; (FIG. 5D) Viability of control (white bars) and +17˜92 cells (black bars) treated for 48 hours with 10 μM (left) or 100 μM (right) Compound 1 with or without 1 mM pyruvate supplementation as measured by flow cytometry; (FIG. 5E) Growth curve of control (● and ▪) and +17˜92 (▴ and ▾) cells cultured with (▪ and ▾) or without (● and ▴) 1 mM pyruvate supplementation. Statistics for all figures are as follows: *, p<0.05; **, p<0.01; ***, p<0.001.

FIG. 6A to FIG. 6B are a series of graphs showing: (FIG. 6A-B) Kaplan-Meier curves of nude mice injected with 1×10⁶ control (A) or +17˜92 (B) lymphoma cells. Mice were provided with 0.9 mg/mL phenformin (n=8), 0.8 mg/mL Compound 1 (n=10), or untreated water (n=10) ad lib. Results are shown for control animals (solid line), phenformine-treated animals (dashed line) and Compound 1-treated animals (dotted lines).

FIG. 7A to FIG. 7E are a series of images and a graph showing: (FIG. 7A) Immunoblot for LKB1 in ﬂ/ﬂ and Δ/Δ cells; (FIG. 7B) Immunoblot of ﬂ/ﬂ and Δ/Δ cells for cleaved caspase 3 following 2 hours treatment with 1 mM phenformin; (FIG. 7C) Immunoblot for LKB1 in Δ/Δ cells expressing LKB1 shRNA; (FIG. 7D) qPCR validation of mature miRNA overexpression following transduction of Eμ-Myc cells with full length miR-17˜92, data presented as relative to mature miRNA expression in Ctrl Eμ-Myc cells; (FIG. 7E) Immunoblot panel of Ctrl and +17˜92 Eμ-Myc cells probing the LKB1-AMPK axis and downstream mTORC1 activation markers.

FIG. 8A to FIG. 8B are a series of graphs showing: (FIG. 8A-B) A 1/3 series dilution of phenformin and Compound 1 applied to Eμ-Myc cells, and OCR (FIG. 8A) and ECAR (FIG. 8B) recorded over time. Drug injection is indicated by the dashed red line.

FIG. 9 is a series of graphs showing: Seahorse trace of Ctrl (blue) and +17˜92 (red) Eμ-Myc cells. Vehicle, 100 μM phenformin, or 10 μM Compound 1 were injected and ECAR (top) and OCR (bottom) were tracked over time. Drug injection is indicated by the dashed line.

FIG. 10A to FIG. 10C are a series of graphs showing: (FIG. 10A) Representative flow plot of control and +17˜92 cells stained with MitoSox mitochondrial ROS dye; (FIG. 10B) Ratios of NAD+/NADH in control (white bars) and +17˜92 (black bars) cells following 2 hours vehicle treatment (−), 100 μM phenformin treatment (P), or 10 μM Compound 1 treatment (I) as measured by LC/MS; (FIG. 10C) Viability of control (white bars) and +17˜92 (black bars) cells treated for 48 hours with 30 μM (left) or 300 μM (right) phenformin with or without 1 mM pyruvate supplementation as measured by flow cytometry. Statistics for all figures are as follows: *, p<0.05; **, p<0.01; ***, p<0.001.

FIG. 11A to FIG. 11C are a series of graphs showing EC₅₀ for phenformin (left panels) and Compound 1 (right panels) for miR-17 (top panels), miR-20 (middle panels), and pri-miR-17 (low panels).

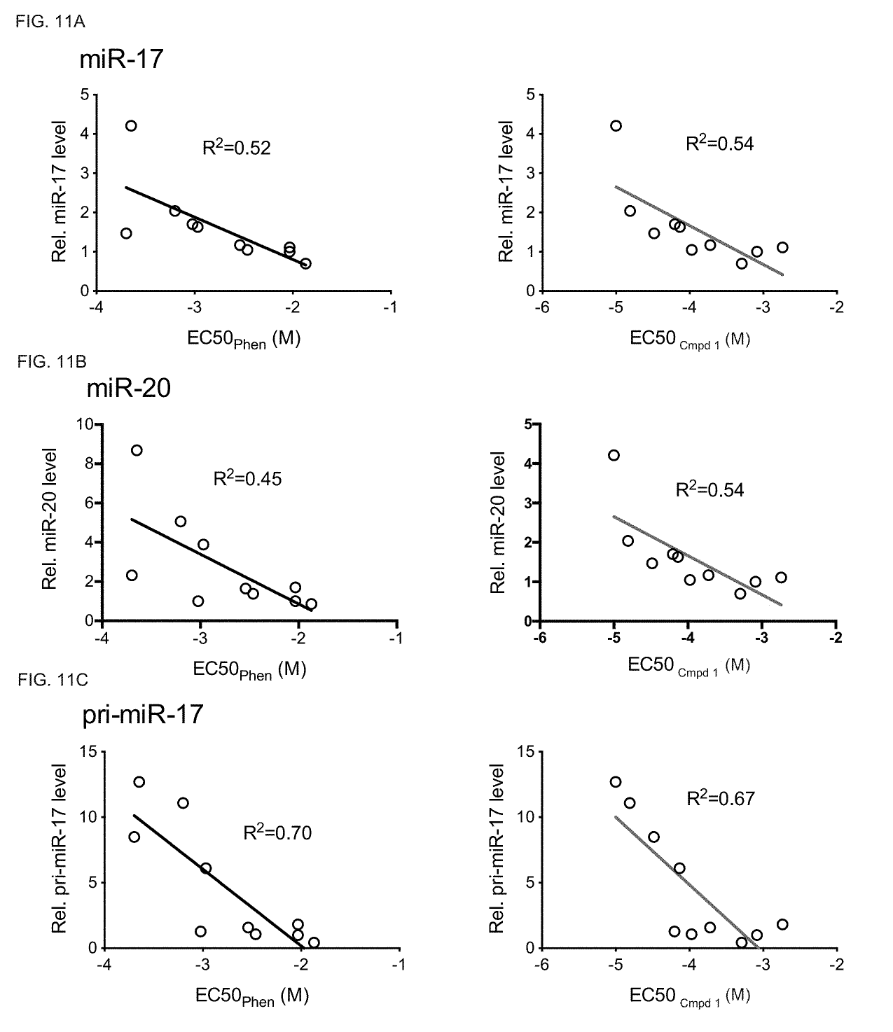

FIG. 12A to FIG. 12D is a series of graphs and images showing (FIG. 12A-B) cell viability for various cell types after (FIG. 12A) phenformin or (FIG. 12B) Compound 1 treatment, as well as miR-17, miR-20a, and pri-miR17˜92 expression of these cell types (FIG. 12C-D).

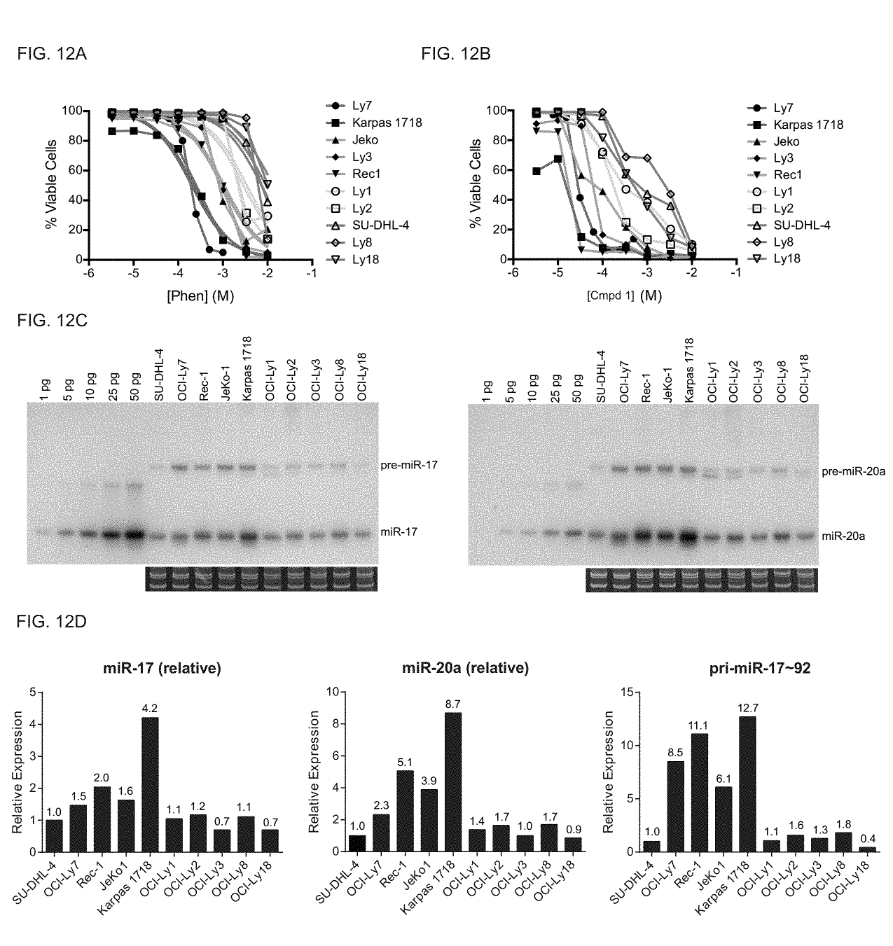

### DETAILED DESCRIPTION

Biomarkers aid in identifying patients who are likely to respond to a therapy and selecting an appropriate treatment for a particular patient. The disclosure provides methods for determining if a patient diagnosed as having cancer is likely to respond to treatment with an effective amount of a biguanide as determined by an increased expression level of miR-17˜92 biomarker. In addition, the disclosure discloses methods for selecting a therapy for a patient being considered for therapy as well as methods of treating cancer in a patient. The present disclosure is based on the discovery that a downstream regulator of Myc-driven metabolism, miR-17˜92, is effective at sensitizing lymphoma cells to biguanide treatment as a consequence of liver kinase B1 (LKB1) inhibition. miR-17˜92 is important in cell cycle, proliferation, apoptosis and other pivotal processes. The miR-17˜92 cluster is very often dysregulated in hematopoietic and solid cancers, cardiovascular, immune and neurodegenerative diseases, and has been implicated in age-related conditions.

Activation of 5′ adenosine monophosphate-activated protein kinase (AMPK) by its upstream LKB1 contributes to the cellular response to energetic stress. LKB1 is a master metabolic regulator whose inactivation in cancer promotes an anabolic metabolic reprogramming at the expense of metabolic flexibility. In the absence of LKB1, AMPK activation is limited and cells are more sensitive to the application of metabolic stress. In a Kras-driven mouse model of non-small cell lung carcinoma (NSCLC), LKB1 loss sensitizes those cancer cells to treatment with phenformin, a biguanide. Whereas LKB1 is frequently lost in NSCLC, it is less frequently found to be mutated or deleted in other cancers. While those NSCLC patients bearing LKB1-null tumors may selectively benefit from biguanide treatment, the limited detection of LKB1 loss in other cancers prevents the widespread use of LKB1 status as a biomarker of biguanide sensitivity.

The polycistronic, oncogenic microRNA (miRNA) cluster miR-17˜92 is a master metabolic regulator downstream of its transcriptional activator, Myc. Myc-driven B lymphoma cells are highly anabolic and aggressively tumorigenic, but deletion of miR-17˜92 in those cells is sufficient to diminish the Myc metabolic phenotype. The miR-17 family is found to be responsible for reinforcing anabolism, and accomplishes this through the inhibition of LKB1 expression. Myc is among the most commonly implicated oncogenes in cancer, and miR-17˜92 itself is found to be overexpressed in a number of cancer types, including those of the colon, lung, and hematopoietic system. Given the extensiveness of Myc and miR-17˜92 expression in cancer, post-transcriptional repression of LKB1 may implicate the tumor suppressor more frequently in cancer than is currently appreciated.

Myc is among the most commonly implicated oncogenes in cancer, and touches upon a broad range of biological processes. Being a transcription factor, small molecule inhibition of Myc has been a persistent challenge, prompting consideration of alternative approaches to Myc inhibition. The metabolic reprogramming orchestrated by Myc has been shown to enforce dependencies on particular metabolic pathways and expose vulnerabilities that can be targeted pharmacologically. As an example, Myc has been demonstrated to render cells dependent on glutaminolysis, and inhibition of glutaminase was shown to act as an effective treatment against Myc-driven cancers.

Detection of miR-17˜92 Biomarker

The biomarker described herein can be detected using any method known in the art. For example, tissue or cell samples from mammals can be conveniently assayed for, e.g., mRNAs or DNAs from the biomarker of interest using Northern, dot-blot, or PCR analysis, array hybridization, RNase protection assay, or using DNA SNP chip microarrays, which are commercially available, including DNA microarray snapshots. For example, real-time PCR (RT-PCR) assays such as quantitative PCR assays are well known in the art.

In some embodiments, expression of miR-17˜92 can be measured by RT-PCR technology. Probes used for PCR may be labeled with a detectable marker, such as, for example, a radioisotope, fluorescent compound, bioluminescent compound, a chemiluminescent compound, metal chelator, or enzyme. Such probes and primers can be used to detect the presence of miR-17˜92 in a sample. As will be understood by the skilled artisan, a great many different primers and probes may be prepared to determine the presence and/or levels of miR-17˜92.

Other methods for determining the level of the biomarker besides RT-PCR or another PCR-based method include proteomics techniques, as well as individualized genetic profiles that are necessary to treat cancer based on patient response at a molecular level. The specialized microarrays herein, e.g., oligonucleotide microarrays or cDNA microarrays, may include the biomarker having expression profiles that correlate with sensitivity to a biguanide treatment. Other methods that can be used to detect nucleic acids, for use in the disclosure, involve high throughput RNA sequence expression analysis, including RNA-based genomic analysis, such as, for example, RNASeq.

Typical protocols for evaluating the status of genes and gene products are found, for example in Ausubel et al. eds., 1995, Current Protocols In Molecular Biology, Units 2 (Northern Blotting), 4 (Southern Blotting), 15 (Immunoblotting) and 18 (PCR Analysis).

### Statistics

The receiver operating characteristic (ROC) curve may be used to determine which subjects will likely be responsive to a biguanide treatment based on their levels of miR-17˜92 biomarker. The ROC curve is generally considered the standard method for describing and assessing the performance of medical diagnostic tests. The ROC curve displays the capacity of a marker to discriminate between two groups of subjects: cases (i.e., subjects with increased levels of the biomarker) versus controls (i.e., normal levels of the biomarker) (Xia et al., Metabolomics. 9(2):280-299 (2013)). The probability of a patient who is likely to benefit from a biguanide treatment includes determining the levels of miR-17˜92 in a patient, generating the ROC curve, and calculating the area under the ROC curve, where area provides the probability of the patient likely benefiting from a biguanide treatment.

### Biguanides

One aspect of the present disclosure provides an N1-cyclic amine-N5-substituted phenyl biguanide derivative compound of Formula I, or a pharmaceutically acceptable salt thereof:

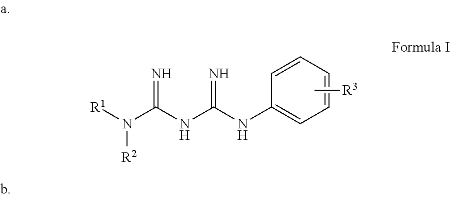

In some embodiments, R¹ and R² are taken together with nitrogen to which they are attached to form 3- to 8-membered heterocycloalkyl, e.g., 3- to 7-membered heterocycloalkyl. Exemplary heterocycloalkyls are azetidinyl, pyrrolidinyl, piperidinyl, morpholinyl, piperazinyl, azepanyl and aziridinyl. In some embodiments, the heterocycloalkyl is unsubstituted or substituted with at least one substituent independently selected from halogen, hydroxyl, and C₁₋₆ alkyl.

R³ may be unsubstituted or substituted and can be unsubstituted hydroxy, substituted C₁₋₆ alkyl, substituted C₁₋₆ alkoxy, unsubstituted or substituted C₁₋₆alkylthio, unsubstituted or substituted amino, unsubstituted or substituted amide, unsubstituted or substituted sulfonamide, nitro, unsubstituted or substituted heteroaryl, cyano, sulfonic acid, and unsubstituted or substituted sulfamoyl, and wherein the substituted R³ has at least one halogen, hydroxyl, or C″ alkyl. An exemplary compound of Formula I is N1-pyrrolidine-N5-(3-trifluoromethoxy)phenyl biguanide.

In some embodiments, a pharmaceutically acceptable salt of compound of Formula I is an acid addition salt of an acid. Exemplary acids include formic acid, acetic acid, propionic acid, lactic acid, butyric acid, isobutyric acid, trifluoroacetic acid, malic acid, maleic acid, malonic acid, fumaric acid, succinic acid, succinic acid monoamide, glutamic acid, tartaric acid, oxalic acid, citric acid, glycolic acid, glucuronic acid, ascorbic acid, benzoic acid, phthalic acid, salicylic acid, anthranyl acid, benzensulfonic acid, p-toluenesulfonic acid, methanesulfonic acid, dichloroacetic acid, aminooxy acetic acid, hydrochloric acid, hydrobromic acid, sulfuric acid, phosphoric acid, nitric acid, carbonic acid, and boric acid.

The biguanides to be used in this disclosure are not limited to the compounds described by Formula I. Other biguanides are listed in U.S. Pat. No. 9,540,325 (e.g., columns 6, 7, and 14-38), U.S. application Ser. No. 14/893,433 (e.g., ¶[0112]-[0284], and Table 1), U.S. Pat. No. 9,539,238 (e.g., column 2), U.S. Pat. No. 9,321,742 (e.g., columns 5, 6, and 12-30), U.S. application Ser. No. 14/766,203 (e.g., ¶[0045]-[0135], and Examples 4-98), and U.S. application Ser. No. 14/528,468 (e.g., ¶[0025]-[0049], ¶[0077]-[0091], ¶[0102]-[0119], ¶[0130]-[0139], ¶[0148]-[0152], ¶[0161]-[0168], ¶[0174]-[0183], and Examples 1-96), each of which is hereby incorporated by reference.

Treatment with a Biguanide

Once the patient population most responsive or sensitive to treatment with the biguanide has been identified, treatment with the biguanide herein, alone or in combination with other medicaments, results in an improvement in the disease. For instance, such treatment may result in a reduction in tumor size or survival (overall, progression free, etc.). Moreover, treatment with the combination of a biguanide herein and at least one second medicament(s) preferably results in an additive, more preferably synergistic (or greater than additive) therapeutic benefit to the patient. Preferably, in this combination method, the timing between at least one administration of the second medicament and at least one administration of the biguanide herein is about one month or less, more preferably, about two weeks or less. Administration of a biguanide, as described herein, is optionally included in the disclosure. Thus, in a further embodiment, the disclosure provides a method of treating cancer (e.g., a bladder cancer, a breast cancer, a colon cancer, a rectal cancer, a uterine cancer, a kidney cancer, leukemia, a liver cancer, a lung cancer, a skin cancer, a hematopoietic system cancer (such as lymphoma), a pancreatic cancer, a prostate cancer, a gastric cancer, a brain cancer, and a thyroid cancer) in a patient by administration of a biguanide (e.g., Compound 1 (i.e., N1-pyrrolidine-N5-(3-trifluoromethoxy)phenyl biguanide)), where the patient is or has been identified as being one that will benefit from such treatment, according to the methods described herein.

It will be appreciated by one of skill in the medical arts that the exact manner of administering to a patient a therapeutically effective amount of a biguanide following a diagnosis of a patient's likely responsiveness to the biguanide will be at the discretion of the attending physician. The mode of administration, including dosage, combination with other agents, timing and frequency of administration, and the like, may be affected by the diagnosis of a patient's likely responsiveness to such biguanide, as well as the patient's condition and history. Thus, even patients diagnosed with cancer who are predicted to be relatively insensitive to the biguanide may still benefit from treatment therewith, particularly in combination with other agents, including agents that may alter a patient's responsiveness to the biguanide.

The composition including a biguanide will be formulated, dosed, and administered in a fashion consistent with good medical practice. Factors for consideration in this context include the particular type of cancer being treated, the clinical condition of the individual patient, the cause of the disease, the site of delivery of the agent, possible side-effects, the type of biguanide, the method of administration, the scheduling of administration, and other factors known to medical practitioners. The effective amount of the biguanide to be administered will be governed by such considerations.

As a general proposition, the effective amount of the biguanide administered parenterally per dose will be in the range of about 20 mg to about 5000 mg, by one or more dosages. Exemplary dosage regimens for biguanides such as Compound 1 include 100 or 400 mg every 1, 2, 3, or 4 weeks or is administered a dose of about 1, 3, 5, 10, 15, or 20 mg/kg every 1, 2, 3, or 4 weeks. The dose may be administered as a single dose or as multiple doses (e.g., 2 or 3 doses), such as infusions.

However, these suggested amounts of a biguanide are subject to a great deal of therapeutic discretion. The key factor in selecting an appropriate dose and scheduling is the result obtained, as indicated above. In some embodiments, the biguanide is administered as close to the first sign, diagnosis, appearance, or occurrence of the disease as possible.

### Pharmaceutical Compositions

In some embodiments, a biguanide is administered as a pharmaceutical composition. The biguanide can be administered by any suitable means, including parenteral, topical, subcutaneous, intraperitoneal, intrapulmonary, intranasal, and/or intralesional administration. Parenteral infusions include intramuscular, intravenous (i.v.), intraarterial, intraperitoneal, or subcutaneous administration. Intrathecal administration is also contemplated. In addition, the biguanide may suitably be administered by pulse infusion, e.g., with declining doses of the biguanide. Preferably, the dosing is given intravenously or subcutaneously, and more preferably by intravenous infusion(s).

If multiple exposures of biguanide are provided, each exposure may be provided using the same or a different administration means. In one embodiment, each exposure is by intravenous administration. In another embodiment, each exposure is given by subcutaneous administration. In yet another embodiment, the exposures are given by both intravenous and subcutaneous administration.

Therapeutic formulations of the biguanides used in accordance with the present disclosure are prepared for storage by mixing the biguanide having the desired degree of purity with optional pharmaceutically acceptable carriers, excipients, or stabilizers in the form of lyophilized formulations or aqueous solutions. For general information concerning formulations, see, e.g., Gilman et al., (eds.) (1990), The Pharmacological Bases of Therapeutics, 8th Ed., Pergamon Press, A Gennaro (ed.), Remington's Pharmaceutical Sciences, 18th Edition, (1990), Mack Publishing Co., Eastori, Pa., Avis et al., (eds.) (1993) Pharmaceutical Dosage Forms: Parenteral Medications Dekker, New York; Lieberman et al., (eds.) (1990) Pharmaceutical Dosage Forms: Tablets Dekker, New York; and Lieberman et al., (eds.) (1990), Pharmaceutical Dosage Forms: Disperse Systems Dekker, New York, Kenneth A Walters (ed.) (2002) Dermatological and Transdermal Formulations (Drugs and the Pharmaceutical Sciences), Vol 119, Marcel Dekker.

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™, or polyethylene glycol (PEG).

The formulation herein may also contain more than one active compound (a second medicament as noted above), preferably those with complementary activities that do not adversely affect each other. The type and effective amounts of such medicaments depend, for example, on the amount and type of a biguanide present in the formulation, and clinical parameters of the subjects. The preferred such second medicaments are noted above.

The active ingredients may also be entrapped in microcapsules prepared, for example, by coacervation techniques or by interfacial polymerization, for example, hydroxymethylcellulose or gelatin microcapsules and poly(methylmethacylate) microcapsules, respectively, in colloidal drug delivery systems (for example, liposomes, albumin microspheres, microemulsions, nanoparticles and nanocapsules) or in macroemulsions. Such techniques are disclosed in Remington's Pharmaceutical Sciences 16th edition, Osol, A Ed. (1980).

Sustained release preparations may be prepared. Suitable examples of sustained release preparations include semi-permeable matrices of solid hydrophobic polymers containing the biguanide, which matrices are in the form of shaped articles, e.g., films, or microcapsules. Examples of sustained release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The formulations to be used for in vivo administration must be sterile. This is readily accomplished by filtration through sterile filtration membranes.

### Dosages

A clinician may use any of several methods known in the art to measure the effectiveness of a particular dosage scheme of a biguanide. For example, in vivo imaging (e.g., MRI) can be used to determine the tumor size and to identify any metastases to determine relative effective responsiveness to the therapy. Dosage regimens may be adjusted to provide the optimum desired response (e.g., a therapeutic response). For example, a dose may be administered, several divided doses may be administered over time or the dose may be proportionally reduced or increased as indicated by exigencies of the therapeutic situation.

A physician having ordinary skill in the art can readily determine and prescribe the effective amount of the pharmaceutical composition required, depending on such factors as the biguanide type. For example, the physician could start with doses of such biguanide, such as Compound 1, employed in the pharmaceutical composition at levels lower than that required in order to achieve the desired therapeutic effect and gradually increase the dosage until the desired effect is achieved. The effectiveness of a given dose or treatment regimen of the biguanide can be determined, for example, by assessing signs and symptoms in the patient using standard measures of efficacy.

In yet another embodiment, the subject is treated with the same biguanide, such as Compound 1 at least twice. Thus, the initial and second biguanide exposures are preferably with the same biguanide, and more preferably all biguanide exposures are with the same biguanide, i.e., treatment for the first two exposures, and preferably all exposures, is with one type biguanide, for example, Compound 1.

In the compositions and methods of the present disclosure, the biguanide (such as Compound 1) may be conjugated with another molecule for further effectiveness, such as, for example, to improve half-life.

In another embodiment, the biguanide (e.g., Compound 1) is the only medicament administered to the subject.

In one embodiment, the biguanide is Compound 1 that is administered at a dose of about 100 or 400 mg every 1, 2, 3, or 4 weeks or is administered a dose of about 1, 3, 5, 10, 15, or 20 mg/kg every 1, 2, 3, or 4 weeks. The dose may be administered as a single dose or as multiple doses (e.g., 2 or 3 doses), such as infusions.

In yet another aspect, the disclosure provides, after the diagnosis step, a method of determining whether to continue administering a biguanide (e.g., Compound 1) to a subject with a cancer, including measuring reduction in tumor size, using imaging techniques, such as radiography and/or MRI, after administration of the biguanide a first time, measuring reduction in tumor size in the subject, using imaging techniques such as radiography and/or MRI after administration of the biguanide a second time, comparing imaging findings in the subject at the first time and at the second time, and if the score is less at the second time than at the first time, continuing administration of the biguanide.

In a still further embodiment, a step is included in the treatment method to test the subject's response to treatment after the administration step to determine that the level of response is effective to treat cancer. For example, a step is included to test the imaging (radiographic and/or MRI) score after administration and compare it to baseline imaging results obtained before administration to determine if treatment is effective by measuring if, and by how much, it has been changed. This test may be repeated at various scheduled or unscheduled time intervals after the administration to determine maintenance of any partial or complete remission.

In one embodiment of the disclosure, no other medicament than a biguanide such as Compound 1 is administered to the subject to treat a cancer.

In any of the methods herein, the biguanide may be administered in combination with an effective amount of a second medicament (where the biguanide (e.g., Compound 1) is a first medicament). Suitable second medicaments include, for example, an anti-neoplastic agent, a chemotherapeutic agent, a growth inhibitory agent, a cytotoxic agent, or combinations thereof.

All these second medicaments may be used in combination with each other or by themselves with the first medicament, so that the expression “second medicament,” as used herein, does not mean it is the only medicament in addition to the first medicament. Thus, the second medicament need not be a single medicament, but may constitute or include more than one such drug.

These second medicaments as set forth herein are generally used in the same dosages and with administration routes as used hereinbefore or about from 1 to 99% of the heretofore-employed dosages. If such second medicaments are used at all, preferably, they are used in lower amounts than if the first medicament were not present, especially in subsequent dosings beyond the initial dosing with the first medicament, so as to eliminate or reduce side effects caused thereby.

For the re-treatment methods described herein, where a second medicament is administered in an effective amount with a biguanide exposure, it may be administered with any exposure, for example, only with one exposure, or with more than one exposure. In one embodiment, the second medicament is administered with the initial exposure. In another embodiment, the second medicament is administered with the initial and second exposures. In a still further embodiment, the second medicament is administered with all exposures. It is preferred that after the initial exposure, such as of steroid, the amount of such second medicament is reduced or eliminated so as to reduce the exposure of the subject to an agent with side effects such as prednisone, prednisolone, methylprednisolone, and cyclophosphamide.

The combined administration of a second medicament includes co-administration (concurrent administration), using separate formulations or a single pharmaceutical formulation, and consecutive administration in either order, wherein preferably there is a time period while both (or all) active agents (medicaments) simultaneously exert their biological activities.

In one embodiment, the biguanide, such as Compound 1, is administered as a slow intravenous infusion rather than an intravenous push or bolus. For example, a steroid such as prednisolone or methylprednisolone (e.g., about 80-120 mg i.v., more specifically about 100 mg i.v.) is administered about 30 minutes prior to any infusion of the biguanide. The biguanide is, for example, infused through a dedicated line.

For the initial dose of a multi-dose exposure to a biguanide, or for the single dose if the exposure involves only one dose, such infusion is preferably commenced at a rate of about 50 mg/hour. This may be escalated, e.g., at a rate of about 50 mg/hour increments every about 30 minutes to a maximum of about 400 mg/hour. However, if the subject is experiencing an infusion-related reaction, the infusion rate is preferably reduced, e.g., to half the current rate, e.g., from 100 mg/hour to 50 mg/hour. Preferably, the infusion of such dose of a biguanide (e.g., an about 1000-mg total dose) is completed at about 255 minutes (4 hours 15 min.). Optionally, the subjects receive a prophylactic treatment of acetaminophen/paracetamol (e.g., about 1 g) and diphenhydramine HCl (e.g., about 50 mg or equivalent dose of similar agent) by mouth about 30 to 60 minutes prior to the start of an infusion.

If more than one infusion (dose) of a biguanide is given to achieve the total exposure, the second or subsequent biguanide infusions in this infusion embodiment are preferably commenced at a higher rate than the initial infusion, e.g., at about 100 mg/hour. This rate may be escalated, e.g., at a rate of about 100 mg/hour increments every about 30 minutes to a maximum of about 400 mg/hour. Subjects who experience an infusion-related reaction preferably have the infusion rate reduced to half that rate, e.g., from 100 mg/hour to 50 mg/hour. Preferably, the infusion of such second or subsequent dose of a biguanide (e.g., an about 1000-mg total dose) is completed by about 195 minutes (3 hours 15 minutes).

In one embodiment, the subject has never been previously administered any drug(s) to treat cancer. In another embodiment, the subject or patient has been previously administered one or more medicaments(s) to treat cancer. In a further embodiment, the subject or patient was not responsive to one or more of the medicaments that had been previously administered. Such drugs to which the subject may be non-responsive include, for example, anti-neoplastic agents, chemotherapeutic agents, cytotosic agents, and/or growth inhibitory agents.

A sample (e.g., blood or tissue biopsy) can be provided from one or more patients before treatment with a biguanide (e.g., Compound 1). The samples may be pooled or maintained as individual samples. The expression of miR-17-92 is assessed in a sample using qPCR. Patients whose samples exhibit an increased expression level of miR-17˜92, e.g., a two-fold increase in expression of miR-17˜92, relative to a control, as described herein, are identified as patients likely to be responsive to treatment with a biguanide.

According to the methods disclosed herein, a physician of skill in the art can treat a patient, such as a human patient having cancer (e.g., a bladder cancer, a breast cancer, a colon cancer, a rectal cancer, a uterine cancer, a kidney cancer, leukemia, a liver cancer, a lung cancer, a skin cancer, a hematopoietic system cancer (such as lymphoma), a pancreatic cancer, a prostate cancer, a gastric cancer, a brain cancer, and a thyroid cancer), so as to inhibit cancer growth, reduce tumor burden, or slow disease progression.

To this end, a physician of skill in the art administers to the human patient an effective amount of Compound 1. Compound 1 can be administered locally (e.g., injected intratumorally) to decrease cancer growth. Compound 1 is administered in a therapeutically effective amount, such as from 1 mg/kg to 20 mg/kg. In some embodiments, Compound 1 is administered bimonthly, once a month, once every two weeks, or at least once a week or more (e.g., 1, 2, 3, 4, 5, 6, or 7 times a week or more). Compound 1 is administered to the patient in an amount sufficient to decrease tumor growth, decrease tumor burden, or increase progression-free survival by 10% or more (e.g., 10%, 20%, 30%, 40%, 50%, 60%, 70%, 80%, 90%, 95% or more). Tumor growth and tumor burden are assessed using standard imaging methods (e.g., digital radiography, positron emission tomography (PET) scan, computed tomography (CT) scan, or magnetic resonance imaging (MRI) scan). Images from before and after administration of Compound 1 are compared to evaluate the efficacy of the treatment, and the rate of disease progression is assessed by comparison to the patient's medical history prior to administration of Compound 1. A finding of a reduction in the total number of tumors, number of primary tumors, volume of tumors, growth of tumors, or rate of disease progression indicates that Compound 1 has successfully treated the cancer.

### Kits

For use in detection of the miR-17˜92 biomarker, kits or articles of manufacture are also provided by the disclosure. Such kits can be used to determine if a patient diagnosed as having cancer is likely to respond to treatment with an effective amount of a biguanide. The kit may include instructions for use of qPCR to determine an expression level of miR-17˜92. An increase in the expression level of miR-17˜92 relative to a reference level expression level of miR-17˜92 may indicate that the patient is likely to respond to treatment with the effective amount of the biguanide.

These kits may include a carrier means being compartmentalized to receive in close confinement one or more container means such as vials, tubes, and the like, each of the container means including one of the separate compounds or elements to be used in the method. For example, one of the container means may include a probe that is or can be detectably labeled. Such probe may be a polypeptide (e.g., an antibody) or polynucleotide specific for a protein or message, respectively. Where the kit utilizes nucleic acid hybridization to detect the target nucleic acid, the kit may also have containers containing nucleotide(s) for amplification of the target nucleic acid sequence (e.g., PCR primers) and/or a container including a reporter-means, such as a biotin-binding protein, e.g., avidin or streptavidin, bound to a reporter molecule, such as an enzymatic, florescent, or radioisotope label.

Such kit will typically include the container described above and one or more other containers including materials desirable from a commercial and user standpoint, including buffers, diluents, filters, needles, syringes, and package inserts with instructions for use. A label may be present on the container to indicate that the composition is used for a specific application, and may also indicate directions for either in vivo or in vitro use, such as those described above.

The kits of the disclosure have a number of embodiments. A typical embodiment is a kit including a container, a label on the container, and a composition contained within the container, wherein the composition includes a primary antibody that binds to a protein or autoantibody biomarker, and the label on the container indicates that the composition can be used to evaluate the presence of such proteins or antibodies in a sample, and wherein the kit includes instructions for using the antibody for evaluating the presence of biomarker proteins in a particular sample type. The kit can further include a set of instructions and materials for preparing a sample and applying antibody to the sample. The kit may include both a primary and secondary antibody, wherein the secondary antibody is conjugated to a label, e.g., an enzymatic label.

Another embodiment is a kit including a container, a label on the container, and a composition contained within the container, wherein the composition includes one or more polynucleotides that hybridize to a complement of miR-17˜92 biomarker under stringent conditions, and the label on the container indicates that the composition can be used to evaluate the presence of miR-17˜92 biomarker in a sample, and wherein the kit includes instructions for using the polynucleotide(s) for evaluating the presence of the biomarker RNA or DNA in a particular sample type.

Other optional components of the kit include one or more buffers (e.g., block buffer, wash buffer, substrate buffer, etc.), other reagents such as substrate (e.g., chromogen) that is chemically altered by an enzymatic label, epitope retrieval solution, control samples (positive and/or negative controls), control slide(s), etc. Kits can also include instructions for interpreting the results obtained using the kit.

### EXAMPLES

### Example 1. Cells Engineered to Overexpress miR-17˜92 Become Highly Sensitized to Phenformin and Compound 1

Human and mouse lymphoma cells engineered to overexpress miR-17˜92 become highly sensitized to phenformin and a biguanide, Compound 1. Both phenformin and Compound 1 are effective promoters of AMPK activation, but miR-17˜92 overexpression is sufficient to abrogate the phosphorylation of AMPK. Biguanide treatment significantly reduces tricarboxylic acid (TCA) cycle intermediate abundances, and, notably, diminishes aspartate levels in +17˜92 cells. Aspartate production fueled by TCA intermediates is an essential function of the TCA cycle in support of proliferation and viability, and the degree of biguanide sensitivity displayed by cells is reflected in the severity of aspartate reduction, possibly as a consequence of diminished purine and pyrimidine biosynthesis. Pyruvate supplementation provides some protection against biguanides in +17˜92 cells but not control cells. This suggests that overexpression of miR-17˜92 imposes more stringent requirements for nicotinamide adenine dinucleotide (NAD+) regeneration.

### Example 2. miR-17˜92 Status Influences Biguanide Sensitivity of Lymphoma Cells

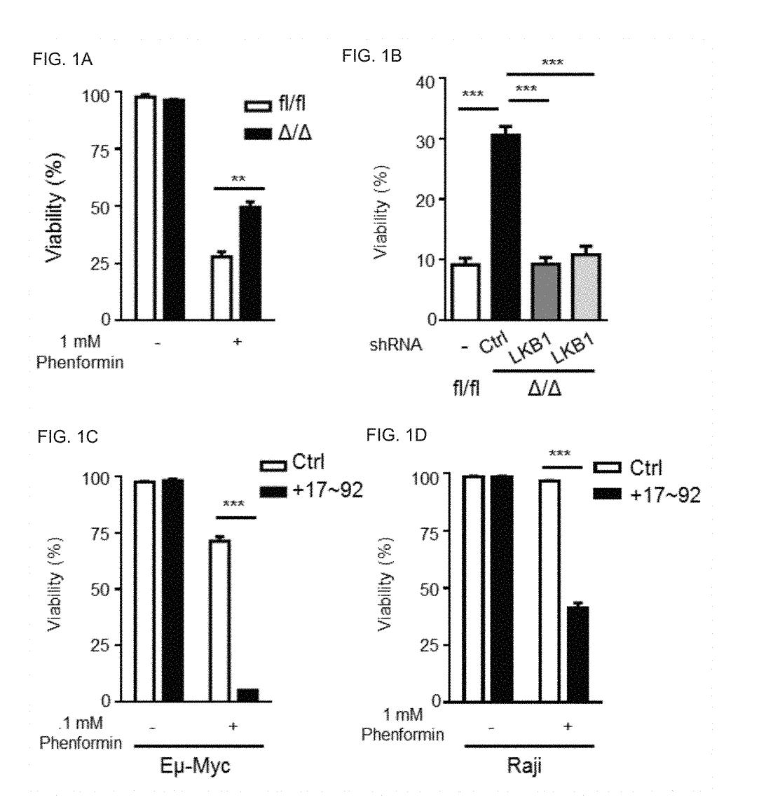

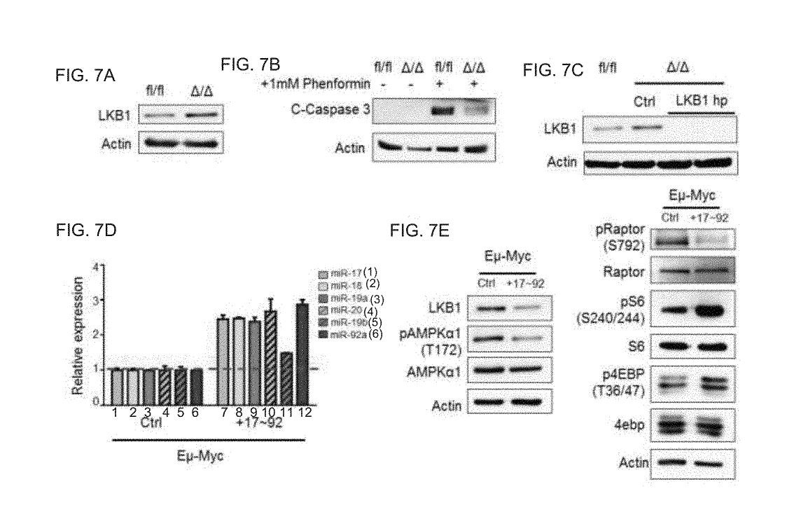

miR-17˜92 cooperates with its transcriptional activator Myc to promote tumorigenesis and anabolic metabolism. To study those biological processes downstream of Myc that remain dependent on miR-17˜92 expression, Eμ-Myc B cell lymphoma cells harboring floxed miR-17˜92 alleles were employed. These cells allow for the conditional deletion of miR-17˜92 in the presence of constitutive Myc expression. The deletion of miR-17˜92 relieves repression on LKB1 expression (FIG. 7A). Treatment of cells with miR-17˜92 intact (ﬂ/ﬂ) and cells deleted for miR-17˜92 (Δ/Δ) with phenformin revealed that those cells lacking miR-17˜92 were more resistant to phenformin treatment (FIG. 1A, FIG. 7B), suggesting that alterations in miR-17˜92 status is sufficient to alter resistance to mitochondrial inhibition.

Δ/Δ cells display much reduced glycolytic and oxidative metabolism, in addition to being weakly tumorigenic when injected into mice (Izreig et al., 2016). The deficiencies in metabolic and tumorigenic activity of Δ/Δ cells can be recovered by shRNA knockdown of LKB1 in those cells. Given that LKB1-null NSCLC cells display increased sensitivity to phenformin, it was tested whether the increase in LKB1 expression mediated the resistance observed in Δ/Δ cells. It was observed that knockdown of LKB1 in Δ/Δ cells abolished the difference in biguanide sensitivity between those cells expressing and lacking miR-17˜92 (FIG. 1B, FIG. 7C).

miR-17˜92 was initially described as being recurrently amplified in lymphoma, and subsequently the tendency for miR-17˜92 overexpression in a range of cancer types was established. To test whether overexpression of miR-17˜92 was sufficient for reducing tolerance of cancer cells to biguanides, Eμ-Myc lymphoma cells were transduced with a vector carrying the entire miR-17˜92 polycistron (“+17˜92 cells”) and overexpression of the mature miRNAs was verified (FIG. 7D). Overexpression of miR-17˜92 produced a reduction in LKB1 expression and enhanced mTORC1 (FIG. 7E). When treated with phenformin, +17˜92 Eμ-Myc lymphoma cells were significantly more sensitive than controls (FIG. 1C). A similar observation was made using a human Burkitt's lymphoma cell line, Raji, transduced to overexpress miR-17˜92 (FIG. 1D). These data suggest that alteration of miR-17˜92 expression influences the sensitivity of cancer cells to biguanide treatment.

### Example 3. Compound 1 is a Novel Biguanide that Inhibits Mitochondrial Respiration

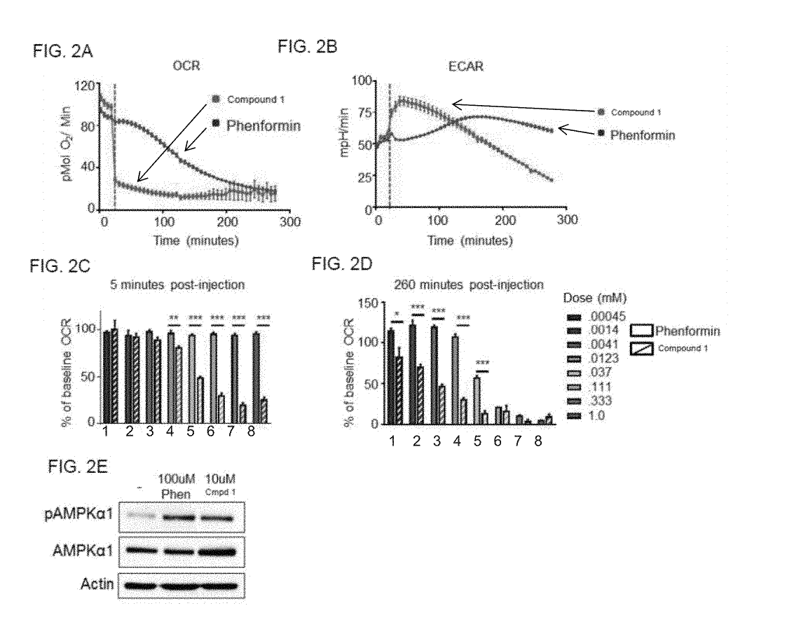

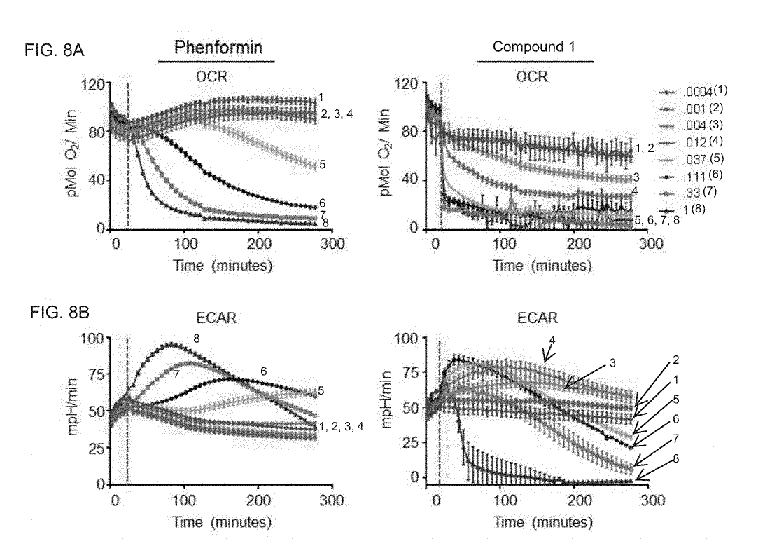

The bioavailability of metformin and its dependence on OCT1 for cellular uptake potentially limit its applicability in the treatment of cancer. A novel biguanide, Compound 1, is more hydrophobic and potentially more potent than metformin. Compound 1 was metabolically profiled against phenformin, a more lipophilic biguanide than metformin, in order to gauge the metabolic effects of Compound 1 treatment. To test efficacy in respiration inhibition, Eμ-Myc cells were acutely treated with either phenformin or Compound 1 using the Seahorse XF96 extracellular flux analyzer. Across a range of doses, Compound 1 and phenformin decreased oxygen consumption rates (OCR) (FIG. 2A, FIG. 8A) with commensurate increases in extracellular acidification rates (ECAR) (FIG. 2B, FIG. 8B). At higher doses, Compound 1 provoked more rapid reductions in OCR as compared to equivalent doses of phenformin (FIG. 2C). Over longer time periods, lower doses of Compound 1 produced more profound reductions in OCR when compared to equivalent doses of phenformin (FIG. 2D). These data suggest that, as an inhibitor of mitochondrial respiration, Compound 1 acts to inhibit oxygen consumption more rapidly and over a larger range in concentration than phenformin.

It was then tested whether Compound 1 and phenformin similarly activate AMPK. Given the difference in potency of Compound 1 and phenformin, the respective doses were used that produced a roughly 50% decrease in OCR from baseline after two hours treatment (FIG. 8A, FIG. 8B). These doses corresponded to 10 μM and 100 μM for Compound 1 and phenformin, respectively. Two hour treatment of Compound 1 or phenformin yielded similar levels of AMPK phosphorylation, demonstrating that Compound 1 can serve as an AMPK activator (FIG. 2E).

### Example 4. miR-17˜92 Overexpression Impairs AMPK Activation and Metabolism Following Biguanide Treatment

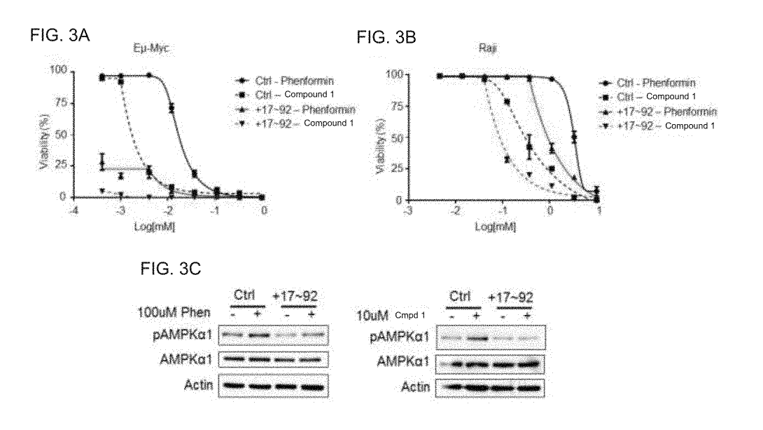

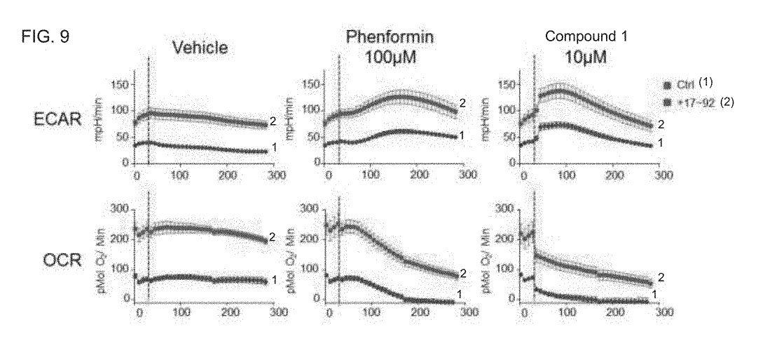

Given that Compound 1 acts as a more potent analogue to phenformin, it was addressed whether miR-17˜92 expression could alter sensitivity to these two drugs over a range of doses. Compound 1 was cytotoxic at lower doses in both control and +17˜92 Eμ-Myc lymphoma cells (FIG. 3A). Notably, at doses of phenformin or Compound 1 that did not elicit any toxicity in control cells, the viability of +17˜92 cells was affected (FIG. 3A). Human Raji cells with or without miR-17˜92 overexpression likewise followed a similar trend, albeit at higher doses of phenformin and Compound 1 (FIG. 3B). Interestingly, at the doses of phenformin and Compound 1 used in these experiments, oxygen consumption of +−17˜92 cells was never eliminated after roughly four hours of treatment, whereas control cells were effectively non-respiring (FIG. 9A). The OCR and ECAR of both cell lines displayed otherwise similar responsiveness to phenformin and Compound 1 (FIG. 9A).

Given that miR-17˜92 overexpression led to LKB1 repression (FIG. 7D), it was tested whether AMPK activation was differentially engaged downstream of phenformin or Compound 1 treatment in control and +17˜92 cells. Following 100 μM phenformin treatment for two hours, +17˜92 cells displayed reduced AMPK phosphorylation than control cells (FIG. 3C). Similarly, AMPK phosphorylation in +17˜92 cells treated with 10 μM Compound 1 for two hours was largely unaffected (FIG. 3C). These data demonstrate that insensitivity of the LKB1-AMPK axis to metabolic stress is engendered by overexpression of miR-17˜92.

### Example 5. Biguanides Affect Central Carbon Metabolism in miR-17˜92 Amplified Cells

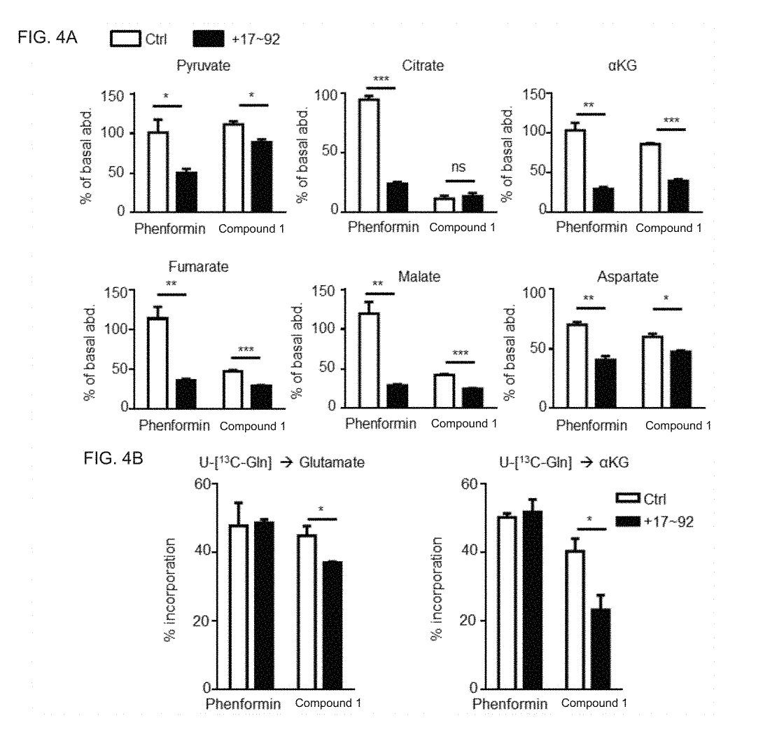

Biguanides are known to reduce TCA cycle intermediate abundances. Thus, the consequences of biguanide treatment on metabolite pools in control and +17˜92 cells were considered. Following two hours of biguanide treatment, +17˜92 cells displayed significant reductions in intracellular pyruvate and TCA cycle intermediate abundances when compared against controls (FIG. 4A).

Aspartate is a proteinogenic amino acid and a substrate for purine and pyrimidine biosynthesis. Recent evidence suggests that a key function of the TCA cycle is the production of aspartate in support of proliferation and viability, and that biguanides are effective suppressors of aspartate biosynthesis (Birsoy et al., 2015). We observed that both phenformin and Compound 1 reduced aspartate abundance in +17˜92 and control cells, with +17˜92 cells experiencing larger reductions in aspartate from baseline than controls (FIG. 4A). These data suggest that +17˜92 cells experience more severe metabolic disruption following biguanide treatment.

In Myc-driven lymphoma, glutamine is a major substrate for TCA cycle anapleurosis. Using a uniformly labelled ¹³C-glutamine tracer, it was tested whether anapleurotic glutamine incorporation into the TCA cycle was differentially effected in control and +17˜92 cells upon biguanide treatment. Compound 1 treatment, but not phenformin treatment, of +17˜92 cells a more significant reduction of ¹³C-glutamine incorporation into glutamate and α-ketoglutarate than was observed in treated control cells (FIG. 4B).

### Example 6. Overexpression of miR-17˜92 Potentiates Oxidative Stress Upon Biguanide Treatment

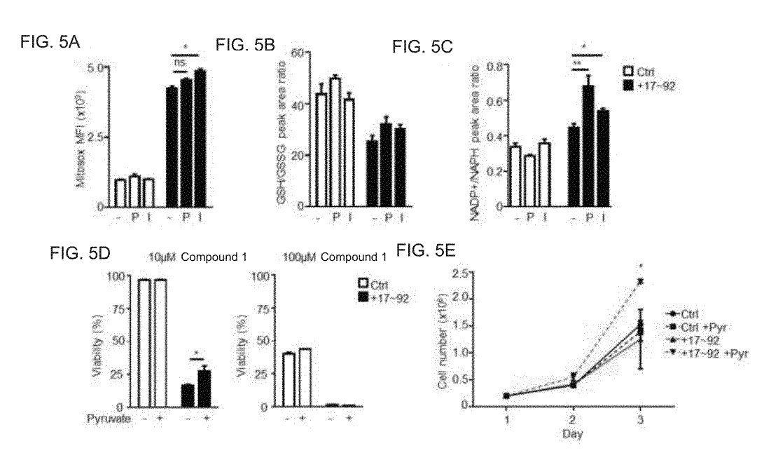

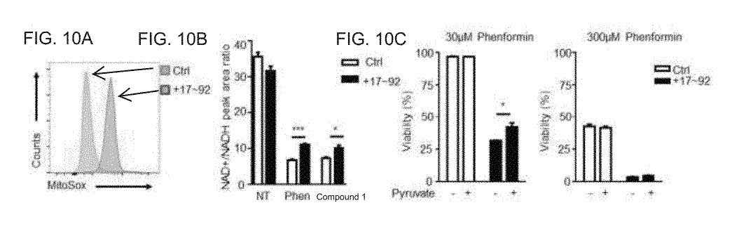

In addition to the biosynthetic and bioenergetic functions of mitochondria, mitochondrial ROS production is relevant to the healthy and pathological operation of a cell. Whereas pharmacological blockade of the electron transport chain (ETC) has been shown to promote ROS generation, conflicting observations regarding the ability of biguanides to increase ROS levels have been reported. The degree to which ROS generation was relevant to the observed differences in sensitivity to biguanide treatment between control and +17˜92 cells was tested. At a baseline, It was observed that +17˜92 cells bear more mitochondrial ROS than controls (FIG. 5A, FIG. 10A). Whereas neither phenformin nor Compound 1 treatment were sufficient to increase mitochondrial ROS levels in control cells, Compound 1 treatment of +17˜92 cells significantly increased mitochondrial ROS, while phenformin treatment approached significance (p=0.053, FIG. 5A). Increased ROS production may, therefore, be a contributor to the enhanced sensitivity to biguanides observed in +17˜92 cells.

Glutathione (GSH) is a key component in the cellular management of ROS. As oxidative stress mounts within a cell, the reduced (GSH) versus oxidized (GSSG) ratio decreases. Using tandem liquid chromatography-mass spectroscopy, it was verified that at a baseline +17˜92 cells possess a lower GSH/GSSG ratio than controls, in agreement with measurements of mitochondrial ROS (FIG. 5A, FIG. 5B). Phenformin and Compound 1 treatment produced no significant changes in the GSH/GSSG ratio (FIG. 5B). The regeneration of GSH from GSSG is catalyzed by glutathione reductase which utilizes NADPH as a cofactor. Reduction of GSSG to produce GSH is, therefore, expected to yield an increase in the NADP+/NADPH ratio. It was found that +17˜92 cells, but not control cells, increase their NADP+/NADPH ratio upon either phenformin or Compound 1 treatment (FIG. 5C). These data suggest that biguanide treatment of +17˜92 cells provoke ROS accumulation in spite of engagement of antioxidant pathways.

A natural consequence of complex I inhibition is the blockade of a route by which NADH may be oxidized to regenerate NAD+. The importance of NAD+ regeneration in maintaining viability upon biguanide treatment was demonstrated. Biguanides were effective at reducing the NAD+/NADH ratio (FIG. 10B). However, +17˜92 cells retained a higher NAD+/NADH ratio than control cells, possibly due to elevated ECAR and residual OCR observed at the biguanide concentrations used (FIG. 9A, FIG. 10B). In order to test whether NAD+ regeneration remained a factor in determining biguanide sensitivity, biguanide treated cells were supplemented with pyruvate to provide a reductive sink for accumulated NADH. At lower doses of phenformin and Compound 1, +17˜92 cells experienced some protective benefit from pyruvate supplementation that was not apparent at higher concentrations (FIG. 5D, FIG. 10C). Control cells, however, experienced no protective effect from pyruvate at any biguanide concentration (FIG. 5D, FIG. 10C). Even in the absence of biguanides, supplementation of +17˜92 cells with pyruvate enhanced proliferation (FIG. 5E). The observations indicated that the relief from reductive stress provided by pyruvate supplementation was unique to +17˜92 cells.

### Example 7. Biguanides are Effective as Single Agents Against miR-17˜92 Overexpressing Lymphoma

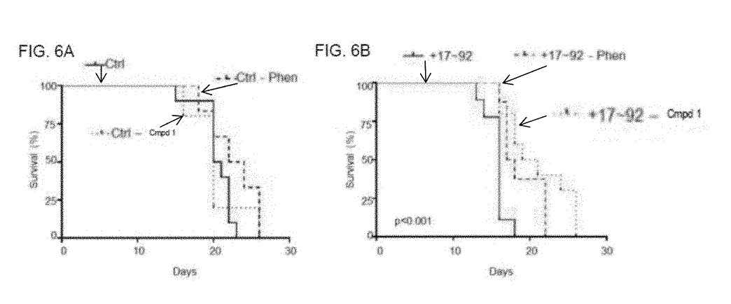

It was determined whether the enhanced sensitivity to biguanides that was observed in vitro extended to in vivo models. Nude mice were injected with either control or +17˜92 cells and phenformin, Compound 1, or untreated water was supplied to mice ad lib and survival was tracked. While biguanide treatment of mice bearing control lymphoma produced no discernable benefit in survival (FIG. 6A), both phenformin and Compound 1 significantly prolonged the lifespan of mice bearing +17˜92 cells (FIG. 6B). Notably, at a baseline those +17˜92 cancer bearing mice succumbed to disease more quickly than control bearing mice, but biguanide treatment was sufficient to extend lifespan to a similar duration as control mice (FIG. 6A, FIG. 6B). The observations indicated that both phenformin and Compound 1 may be suitable agents in treating those cancers with amplified miR-17˜92 expression.

### Example 8. Cell Lines, DNA Constructs, and Cell Culture

The generation of Eμ-Myc Cre-ERT2⁺; miR-17˜92^{ﬂ/ﬂ} lymphoma cells has been described (Mu et al., 2009c). Deletion of miR-17˜92 was achieved by culturing Eμ-Myc Cre-ERT2⁺; miR-17˜92″ cells with 250 nM 4-OHT for four days, followed by subcloning 4-OHT-treated cells to isolate cells deficient for miR-17˜92. Eμ-Myc cells were cultured on a layer of irradiated Ink4a-null MEF feeder cells in DMEM and IMDM medium (50:50 mix) supplemented with 10% fetal bovine serum (FBS), 20000 U/mL penicillin, 7 mM streptomycin, 2 mM glutamine, and β-mercaptoethanol. Raji cells were cultured in RPMI medium supplemented with 10% FBS, 20000 U/mL penicillin, 7 mM streptomycin, and 2 mM glutamine. Cells were grown at 37° C. in a humidified atmosphere supplemented with 5% (v/v) CO₂.

Retroviral-mediated gene transfer into lymphoma cells was conducted. Lymphoma cells were transduced via spin infection, followed by culture in 4 μg/mL puromycin for four days, and subsequent subcloning by limiting dilution. miR-17˜92 constructs have been previously described. Knockdown of Stk11 via shRNA (sequence: 5′-AGGTCAAGATCCTCAAGAAGAA-3′, SEQ ID NO: 1) was achieved using the miR-30-adapted LMP retroviral vector system.

### Example 9. Cell Proliferation and Viability Assay

Cells were seeded at a density of 1×10⁵ cells/mL in 3.5 cm dishes, and cell counts determined via trypan blue exclusion using a TC20 Automated Cell Counter (Biorad). For viability measurements, cells were stained with Fixable Viability Dye eFluor 780 (eBioscience), and analyzed using a Gallios flow cytometer (Beckman Coulter, Fullerton, Calif.) and FlowJo software (Tree Star, Ashland, Oreg.).

### Example 10. Seahorse XF96 Respirometry and Metabolic Assays

Cellular oxygen consumption rate (OCR) and extracellular acidification rate (ECAR) were determined using an XF96 Extracellular Flux Analyzer (Seahorse Bioscience) (Faubert et al., 2014b; Vincent et al., 2015a). 7.5×10⁴ lymphoma cells were plated per well of an XF96 Seahorse plate in 140 μL of unbuffered DMEM containing 25 mM glucose and 2 mM glutamine, followed by centrifugation at 500×g for five minutes. Seahorse plates were pre-coated with poly-D-lysine (Sigma-Aldrich) to enhance cell adherence. XF assays consisted of sequential mix (3 min), pause (3 min), and measurement (5 min) cycles, allowing for determination of OCR and ECAR every 8 min. Following four baseline measurements, 20 μL of untreated media, phenformin, or Compound 1 were injected into respective wells, and OCR and ECAR tracked over time. For media metabolite determination, cells were seeded at 1×10⁵ cells/mL in 3.5 cm plates, and cultured for two days prior to harvesting medium. Culture medium was analyzed for extracellular metabolites (glucose and lactate) using a BioProfile Analyzer (NOVA Biomedical) and normalized to cell number.

### Example 11. GC-MS Analysis of ¹³C-Labelled Metabolites

Cellular metabolites were extracted and analyzed by GC-MS. Eμ-Myc cells (3-5×10⁶ per 3.5 cm dish) were incubated for 2 hours in untreated, 100 μM phenformin, or 10 μL Compound 1 medium containing 10% dialyzed FBS and [¹³C]-glutamine (Cambridge Isotope Laboratories). Cells were washed twice with saline, then lysed in ice-cold 80% methanol and sonicated. For GC-MS analysis, D-myristic acid (750 ng/sample) was added to metabolite extracts as an internal standard prior to drying samples under a N₂ stream. Dried extracts were dissolved in 30 μL methoxyamine hydrochloride (10 mg/mL) in pyridine and derivatized as tert-butyldimethylsilyl (TBDMS) esters using 70 μL N-(tert-butyldimethylsilyl)-N-methyltrifluoroacetamide (MTBSTFA). An Agilent 5975C GC-MS equipped with a DB-5MS+DG (30 m×250 μm×0.25 μm) capillary column (Agilent J&W, Santa Clara, Calif., USA) was used for all GC-MS experiments, and data collected by electron impact set at 70 eV. A total of 1 μL of derivatized sample was injected per run in splitless mode with inlet temperature set to 280° C., using helium as a carrier gas with a flow rate of 1.5512 mL/min (rate at which myristic acid elutes at 17.94 min). The quadrupole was set at 150° C. and the GC/MS interface at 285° C. The oven program for all metabolite analyses started at 60° C. held for 1 min, then increasing at a rate of 10° C./min until 320° C. Bake-out was at 320° C. for 10 min. Sample data were acquired in scan mode (1-600 m/z) (McGuirk et al., 2013). Mass isotopomer distribution for TCA cycle intermediates was determined using a custom algorithm developed at McGill University. After correction for natural ¹³C abundances, a comparison was made between non-labeled (¹²C) and ¹³C-labeled abundances for each metabolite. Metabolite abundance was expressed relative to the internal standard (D-myristic acid) and normalized to cell number.

### Example 12. LC-MS Analysis of NAD+/NADH

All LC/MS grade solvents and salts were purchased from Fisher (Ottawa, Ontario Canada: water (H₂O), acetonitrile (ACN), methanol (MeOH), and formic acid. The authentic metabolite standards were purchase from Sigma-Aldrich Co. (Oakville, Ontario, Canada).

Cultured cells were washed with cold 150 mM ammonium formate solution pH of 7.4 and then extracted with 600 μL of 31.6% MeOH/36.3% ACN in H₂O (v/v). Cells were lysed and homogenized by bead-beating for 2 min at 30 Hz using six 1.4 mm ceramic beads (TissueLyser II—Qiagen). Cellular extracts were partitioned into aqueous and organic layers following dimethyl chloride treatment and centrifugation. Aqueous supernatants were dried by vacuum centrifugation with sample temperature maintained at −4° C. (Labconco, Kansas City Mo., USA). Pellets were subsequently resuspended in 25 μL of H₂O as the injection buffer.

Chromatographic separation was performed on a Scherzo SM-C18 column 3 μm, 3.0×150 mm (Imtakt Corp, JAPAN). The chromatographic gradient started at 100% mobile phase A (0.2% formic acid in water) with a 2 min hold followed with a 6 min gradient to 80% B (0.2% formic acid in MeOH) at a flow rate of 0.4 mL/min. This was followed by a 5 min hold time at 100% mobile phase B and a subsequent re-equilibration time (6 min) before next injection.

Reduced (GSH, m/z: 308.0911) and oxidized (GSSG, m/z: 613.1592) forms of glutathione were measured (area under the curve for metabolite ions) using an Agilent 6540 UHD Accurate-Mass Q-TOF mass spectrometer (Agilent Technologies, Santa Clara, Calif., USA) equipped with a 1290 Infinity ultra-performance LC system (Agilent Technologies). Analyte ionization was accomplished using ESI in positive ionization mode. The source operating conditions were set at 325° C. and 9 l/min for gas temperature and flow respectively, nebulizer pressure was set at 40 psi and capillary voltage was set a 4.0 kV. Reference masses 121.0509 and 922.0099 were introduced into the source through a secondary spray nozzle to ensure accurate mass. MS data were acquired in full scan mode mass range: m/z 100-1000; scan time: 1.4 s; data collection: centroid and profile modes. Retention times, accurate masses, and MS/MS (10, 20, 30, 40 V) for each compound were confirmed against authentic standards.

For all LC/MS analyses, 1 μL of sample was injected. The column temperature was maintained at 10° C.

Data were quantified by integrating the area under the curve of each compound using MassHunter Quant (Agilent Technologies, Santa Clara, Calif., USA). Each metabolite's accurate mass ion was extracted (EIC) using a 10 ppm window. Relative concentrations were determined from external calibration curves.

### Example 13. Immunoblotting and Quantitative Real-Time PCR

Lymphoma cell lines were subjected to SDS-PAGE and immunoblotting using CHAPS and AMPK lysis buffers. Primary antibodies against β-actin, 4EBP (total, phospho-T36/47, and phospho-S65), rS6 (total and p S235/236), Raptor (total and pS792), and AMPKα (total and phospho-T172) were obtained from Cell Signaling Technology (Danvers, Mass.). Primary antibody against LKB1 (Ley 37D/G6) was obtained from Santa Cruz Biotechnology (Dallas, Tex., USA). For qPCR quantification of mature miRNAs, Qiazol was used isolate RNA, miRNEasy Mini kit was used to purify miRNAs and total mRNA, and cDNA synthesized using the miScript II RT kit (Qiagen). Quantitative PCR was performed using the SensiFAST SYBR Hi-ROX kit (Bioline) and an AriaMX Real-Time PCR system (Agilent Technologies). miScript primer assays (Qiagen) were used to detect mature miRNAs of the miR-17˜92 cluster, with miRNA expression normalized relative to U6 RNA levels.

### Example 14. Tumor Xenograft Assays

Lymphoma cells were resuspended in HBSS at a concentration of 5×10⁶ cells/mL, and 10⁶ cells/200 μL were injected intravenously into CD-1 nude mice (Charles River). Water bottles carrying 1.2% sucralose, 0.9 mg/mL phenformin+1.2% sucralose, or 0.8 mg/mL Compound 1+1.2% sucralose were provided for ad lib consumption. Mice were tracked until clinical displays of disease, such as weight loss and poverty of movement, at which point mice were euthanized.

### Example 15. Statistical Analysis

Statistics were determined using paired Student's t-test, ANOVA, or Log-rank (Mantel-Cox) using Prism software (GraphPad). Data were calculated as the mean±SEM for biological triplicates, and the mean±SD for technical replicates unless otherwise stated. Statistical significance was represented in figures by: *, p<0.05; **, p<0.01; ***, p<0.001.

### REFERENCES

Cronin M, Pho M, Dutta D, Stephans J C, Shak S, Kiefer M C, Esteban J M, Baker J B. Measurement of gene expression in archival paraffin-embedded tissues: development and performance of a 92-gene reverse transcriptase-polymerase chain reaction assay. Am J Pathol. 2004 January; 164(1):35-42.
Ma X J, Wang Z, Ryan P D, Isakoff S J, Barmettler A, Fuller A, Muir B, Mohapatra G, Salunga R, Tuggle J T, Tran Y, Tran D, Tassin A, Amon P, Wang W, Wang W, Enright E, Stecker K, Estepa-Sabal E, Smith B, Younger J, Balis U, Michaelson J, Bhan A, Habin K, Baer T M, Brugge J, Haber D A, Erlander M G, Sgroi D C. A two-gene expression ratio predicts clinical outcome in breast cancer patients treated with tamoxifen. Cancer Cell. 2004 June; 5(6):607-16.
Mogilyansky E, Rigoutsos I. The miR-17/92 cluster: a comprehensive update on its genomics, genetics, functions and increasingly important and numerous roles in health and disease. Cell Death Differ. 2013 December; 20(12):1603-14.

What is claimed is:

1. A method of treating cancer in a patient, the method comprising:
(a) detecting expression of miR-17˜92 in a sample obtained from the patient prior to administration of an effective amount of a biguanide;
(b) comparing the expression level of miR-17˜92 to a reference expression level of miR-17˜92, wherein an increase in the level of expression of miR-17˜92 in the patient sample relative to the reference level identifies a patient who is likely to respond to treatment with an effective amount of a biguanide; and
(c) if the patient is identified as being likely to respond to treatment, administering to the patient the effective amount of the biguanide.

2. The method of claim 1, wherein the expression level of miR-17˜92 is increased at least two-fold relative to the reference expression level.

3. The method of claim 1, wherein the reference expression level is the expression level of miR-17˜92 in a reference population or a pre-assigned expression level for miR-17˜92.

4. The method of claim 1, wherein the miR-17˜92 expression level is determined using quantitative polymerase chain reaction (qPCR).

5. The method of claim 1, wherein the cancer is selected from the group consisting of colon cancer, lung cancer, lymphoma and hematopoietic system cancer.

6. The method of claim 1, wherein the effective amount of the biguanide comprises a N1-cyclic amine-N5-substituted biguanide derivative compound of Formula I or a pharmaceutically acceptable salt thereof: 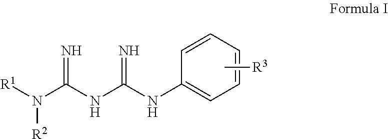
wherein R¹ and R² are taken together with nitrogen to which they are attached to form 3- to 8-membered heterocycloalkyl selected from the group consisting of azetidinyl, pyrrolidinyl, piperidinyl, piperazinyl, azepanyl and aziridinyl, wherein the heterocycloalkyl is unsubstituted or substituted with at least one substituent independently selected from the group consisting of halogen, hydroxy and C₁₋₆ alkyl;
R³ is unsubstituted or substituted and is selected from the group consisting of unsubstituted hydroxy, substituted C₁₋₆ alkyl, substituted C₁₋₆ alkoxy, unsubstituted or substituted C₁₋₆ alkylthio, unsubstituted or substituted amino, unsubstituted or substituted amide, unsubstituted or substituted sulfonamide, nitro, unsubstituted or substituted heteroaryl, cyano, sulfonic acid, and unsubstituted or substituted sulfamoyl, and
wherein the substituted R³ has at least one substituent selected from the group consisting of halogen, hydroxy and C₁₋₆ alkyl.

7. The method of claim 6, wherein the effective amount of the biguanide comprises an N1-cyclic amine-N5-substituted biguanide derivative compound of Formula I or a pharmaceutically acceptable salt thereof:
wherein R¹ and R² are taken together with nitrogen to which they are attached to form 3- to 7-membered heterocycloalkyl selected from the group consisting of azetidinyl, pyrrolidinyl, piperidinyl, piperazinyl, azepanyl and aziridinyl, wherein the heterocycloalkyl is unsubstituted or substituted with at least one substituent independently selected from the group consisting of halogen, hydroxy and C₁₋₆ alkyl;
R³ is unsubstituted or substituted and is selected from the group consisting of unsubstituted hydroxy, substituted C₁₋₆ alkyl, substituted C₁₋₆ alkoxy, unsubstituted or substituted C₁₋₆ alkylthio, unsubstituted or substituted amino, unsubstituted or substituted amide, unsubstituted or substituted sulfonamide, nitro, unsubstituted or substituted heteroaryl, cyano, sulfonic acid, and unsubstituted or substituted sulfamoyl, and
wherein the substituted R³ has at least one substituent selected from the group consisting of halogen, hydroxy and C₁₋₆ alkyl.

8. The method of claim 9, wherein the compound of Formula I is N1-pyrrolidine-N5-(3-trifluoromethoxy)phenyl biguanide, or a pharmaceutically acceptable salt thereof.

9. The method of claim 8, wherein the pharmaceutically acceptable salt is an acid addition salt of an acid selected from the group consisting of formic acid, acetic acid, propionic acid, lactic acid, butyric acid, isobutyric acid, trifluoroacetic acid, malic acid, maleic acid, malonic acid, fumaric acid, succinic acid, succinic acid monoamide, glutamic acid, tartaric acid, oxalic acid, citric acid, glycolic acid, glucuronic acid, ascorbic acid, benzoic acid, phthalic acid, salicylic acid, anthranyl acid, benzensulfonic acid, p-toluenesulfonic acid, methanesulfonic acid, dichloroacetic acid, aminooxy acetic acid, hydrochloric acid, hydrobromic acid, sulfuric acid, phosphoric acid, nitric acid, carbonic acid, and boric acid.

10. A kit for determining whether a patient diagnosed as having cancer is likely to respond to treatment with an effective amount of a biguanide, the kit comprising instructions for use of qPCR to determine an expression level of miR-17˜92, wherein an increase in the expression level of miR-17˜92 relative to a reference level expression level of miR-17˜92 indicates that the patient is likely to respond to treatment with the effective amount of the biguanide.
